(19) United States
(12) Patent Application Publication
(10) Pub. No.: US 2008/0187952 A1
(43) Pub. Date: Aug. 7, 2008

(54) Biomarkers of Ionizing Radiation Response

(75) Inventors: Gabriela G. Cezar, Middleton, WI (US); Alan M. Smith, Madison, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: WISCONSIN ALUMNI RESEARCH FOUNDATION, Madison, WI (US)

(21) Appl. No.: 12/023,868

(22) Filed: Jan. 31, 2008

Related U.S. Application Data
(60) Provisional application No. 60/888,198, filed on Feb. 5, 2007.
(60) Provisional application No. 60/899,715, filed on Feb. 6, 2007.

(51) Int. Cl. C12Q 1/02; C12Q 1/00; G01N 33/48; G01N 33/50

(52) U.S. Cl. 435/29; 435/4; 436/63; 436/129

(57) ABSTRACT

The invention provides methods for measuring cellular response to ionization radiation (IR). The invention also provides a plurality of diagnostic and prognostic cancer biomarkers for assessing cellular response to IR.

This application claims the priority benefit of U.S. provisional patent applications, Ser. Nos. 60/888,198, filed Feb. 5, 2007, and 60/899,715, filed Feb. 6, 2007, the entirety of which are herein incorporated by reference.

### FIELD OF THE INVENTION

This invention provides reagents and methods for assessing cellular response to ionizing radiation, a principal modality in cancer therapy. In particular, the invention provides methods using metabolomics for detecting and assessing the presence of small molecules in irradiated cell populations, in comparison to the presence or absence of said molecules in nonirradiated cells. Specific biomarkers for radiation response identified herein are also provided. Such biomarkers are useful for diagnostic and prognostic indicators of cancer, cancer treatment, tumor response to radiation therapy, and exposure to radiation.

### BACKGROUND OF THE INVENTION

Approximately one-half of all cancer patients receive radiation therapy (www.cancer.gov/cancertopics/factsheet/Therapy/radiation, Jan. 28, 2006). In radiation therapy (also called radiotherapy, x-ray therapy, or irradiation), certain types of ionizing radiation (IR) are used to kill cancer cells and reduce or eliminate tumors. Id. Radiation therapy may be used alone or in combination with other cancer treatments, such as chemotherapy or surgery. In some cases, a patient may receive more than one type of radiation therapy. Id. Radiation therapy may be used to treat almost every type of solid primary or metastatic tumor, including cancers of the brain, breast, cervix, larynx, lung, pancreas, prostate, skin, spine, stomach, uterus, or soft tissue sarcomas. Id. Radiation dose to each site depends on a number of factors, including the type of cancer and whether there are tissues and organs nearby that may be damaged by radiation.

For malignant brain tumors, such as gliomas, radiation therapy is a standard-of-care for intervention primarily because of difficulties associated with delivery of chemotherapeutic agents to the brain. There are an estimated 20,500 cases of newly diagnosed primary brain malignancies per year; glioblastoma multiforme (GBM), the most aggressive form, is also the most common (American Cancer Society, 2007, Cancer Facts & Figures 2007, Atlanta: American Cancer Society). GBM is typically treated using a combined modality approach, consisting of surgery, radiation, and chemotherapy (Peacock & Lesser, 2006, Curr Treat Options Oncol. 7(6):479-89). Radiation therapy, however, remains the primary treatment modality for malignant glioma. Unfortunately, several decades of effort towards improving clinical outcomes of GBM have been largely unsuccessful, with an overwhelming majority of patients recurring locally and rapidly succumbing to uncontrolled disease progression despite delivery of high doses of radiation to tumor. A clinically meaningful survival benefit in GBM has only recently been established by the European Organization for Research and Treatment of Cancer (EORTC) (Stupp et al., 2005, N Engl J Med 352: 987-996), with the addition of the alkylating agent temozolomide to radiation therapy. Despite this progress, local control and long term survival remains frankly dismal and mechanistic understanding of the intrinsic radiation resistance of GBM requires further investigation.

Glial cells are responsible for the support of neurons and have high metabolic activity. Certain small molecule metabolites, measured by non-metabolomic approaches, have been associated with gliomas and thus are markers for malignant glial cells. These include polyunsaturated fatty acids, nucleotides, alanine, glutamate, N-acetylaspartate and choline-containing metabolites (Griffin & Shockcor, 2004, Nat Rev Cancer 4:551-61; McKnight, 2004, Semin Oncol. 31:605-17). Unfortunately the fundamental processes underlying radiation response in malignant gliomas and their intrinsic radiation resistance have not been fully elucidated. This lack of understanding is largely based on the exceedingly complex nature of the radiation response, which consists of the convergence of hundreds of signaling pathways determined by fundamental cellular events. Recent experiments using transcriptomic and proteomic approaches, offer important insights into the complex nature of these interactions by testing tens of thousands of cellular processes in a single experiment (Szkanderova et al., 2005, Radiation Res 163: 307-15; Camphausen et al., 2005, Cancer Res 65: 10389-10393; Khodarev et al., Proc Natl Acad Sci USA 98: 12665-12670).

Although these “omics” platforms are likely to provide valuable insight into the genetic basis of disease process, they are limited by the fact that they usually require tissue for analysis. Characterizing how a tumor responds to a particular cytotoxic insult (i.e. radiation) during therapy requires multiple repeat biopsies and is therefore not clinically feasible. Non-invasive, real-time assessment of tumor response is being actively investigated using imaging, including PET and MR mass-spectroscopy, but these are still exploratory, costly, relatively non-specific, and have limited insight into specific cellular pathways contributing towards resistance (Ott et al., 2006, J Clin Oncol 24: 4692-8; Bezabeh et al., 2005, Am J Neuroradiol 26: 2108-2113; Gillies et al., 2000, Neoplasia 2: 139-151; Evelhoch et al., 2005, Cancer Res 65: 7041-7044; Griffin & Shockcor, 2004, Nat Rev Cancer 4: 551-61).

Metabolomics is the systematic and quantitative analysis of the diverse set of metabolites created through biologically catalyzed reactions. When applied to study pathophysiological changes caused by genetic or noxious agents this holistic examination of metabolic changes becomes a powerful tool to identify biochemical pathways effected by the agent of interest (Nicholson et al., 1999, Xenobiotica 29: 1181-9; Nicholson et al., 2002, Nat Rev Drug Discov 1: 153-61; Fiehn, 2002, Plant Molecular Biology 48: 155-171.). Metabolite biomarkers have benefits over traditional mRNA or protein markers because metabolites are created through the enzymatic action of functional proteins. These functional proteins are a product of mRNA that has been translated into proteins with proper post-translation modifications and the co-factors necessary for in vivo biological activity. Because metabolites are the product of functioning and active biochemical pathways, as biomarkers, they permit the assay of changes in actual active biological processes, processes that may only be predicted in transcriptomic and proteomic studies. Transcriptomic and proteomic studies fail to measure functional biochemical pathways as an endpoint. Metabolomics measures metabolites that are the phenotypic output of functional aspects of many different cellular and organismal processes present in for example, the genome, epigenome, transcriptome, proteome, interactome and signal transduction. One of the most promising aspects of metabolomic studies is that it permits the identification of changes in functional pathways.

Metabolomics may be performed using liquid or gas chromatography coupled to mass spectrometry that permit separation, identification, and quantification of metabolites. This technology can be used for profiling the dynamic set(s) of metabolites present in chemically complex samples such as biofluids, tissues, and media from cancer cell cultures. Metabolites that are altered in reproducible and robust manners in response to pathological or chemical insults in these biological matrices can serve as biomarkers of disease or toxic response (Cezar et al., 2007, Stem Cells and Development 16: 1-14; Griffin & Bollard, 2004, Curr Drug Metab 5: 389-398; van Ravenzwaay et al., 2007, Toxicol Lett 172: 21-8). Thus, metabolite profiling creates functional insight into the biochemical response of cancer to therapy. Differentially affected metabolites can be translated as biomarkers into the clinical setting and assayed in patient biofluids, such as serum, plasma, cerebrospinal fluid, urine, lymph, or saliva to test response to therapy or measure cancer severity.

The analysis of glioma cell lines and cancer cells derived from gliomas has revealed that specific metabolic pathways are involved in the radiation response. This suggests that specific cellular pathways are involved in the susceptibility or refractoriness of these cells to IR. Hence there exists a need in the art to define how tumors respond to radiation and more specifically, why gliomas are resistant to IR therapy. There also exists a need in the art to identify small molecule markers for gliomas that are resistant or sensitive to radiation for diagnosis, prognosis and course-of-treatment monitoring.

Thus, there is a need in the art to identify said biomarkers for improving IR treatment of cancer patients and to serve as a basis for personalized medicine, to increase the efficacy and safety of cancer care according to individual biomarker profiles.

### SUMMARY OF THE INVENTION

This invention provides novel biomarkers specific for ionizing radiation (IR) response and methods for identifying said markers.

In a first aspect, the invention provides methods for identifying biomarkers for IR response in gliomas. In certain embodiments, the biomarkers are identified by metabolomics methods using a glioma cell line, U373 (available from the American Type Culture Collection (ATCC), Manassas, Va. under Accession No. HTB17). In further embodiments, biomarkers are identified in a plurality of glioma cell lines, including but not limited to U373 (ATCC Accession No. HTB17), T98G (ATCC Accession No. CRL-1690), and U251 (provided by Paul Harari, University of Wisconsin-Madison). Biomarkers provided in this aspect are small molecule metabolites produced by said glioma cells in response to IR.

In certain embodiments of this aspect of the invention, these small molecule metabolites are used for clinical monitoring and establishing a prognosis for radiotherapy response. In particular, the prevalence of these candidate biomarkers in patient biofluids (in non-limiting examples, blood and fluid components thereof such as plasma and serum urine, lymph, cerebrospinal fluid, and saliva) prior to and during radiation therapy can inform physicians on the expected outcomes of radiation therapy in individuals, e.g. personalized medicine.

This invention provides methods for measuring cellular response to IR, and for reliably determining the cellular and biochemical effects of ionizing radiation exposure. In this aspect, the invention provides profiles comprising a plurality of small molecule biomarkers specific for irradiated cells, including such profiles that are specific to particular tumor cell types as well as profiles in common between two or a plurality of cell types. In certain aspects, said profiles are provided wherein metabolic profiles are altered in irradiated cells. In particular embodiments, the invention provides a profile of biomarkers from different active metabolic reactions, pathways, and networks whose response is altered by exposure of the cells to ionizing radiation.

In additional aspects, the invention provides methods for metabolomic evaluation of cells exposed to ionizing radiation. In these methods, cells, including malignant cells and particularly glioma cells, are exposed to ionizing radiation, preferably at conventional clinical levels. Following IR treatment, cellular metabolic products are identified in IR-exposed cells and small molecule metabolites identified. In particular, biomarkers are identified in said cells in comparison with nonirradiated cells, wherein metabolic changes consequent to IR treatment are identified. In certain aspects, said comparisons are used to identify metabolic pathways activated or inhibited by IR treatment.

The invention thus provides methods for identifying predictive biomarkers of ionizing radiation response. In certain embodiments of this aspect, a dynamic set representative of a plurality of small molecules present in cells is determined and correlated with health and disease or IR-treatment. Small molecules such as sugars, organic acids, amino acids, fatty acids, and signaling low molecular weight compounds participate in and reveal functional mechanisms of cellular response to pathological or radiation insult, thus serving as biomarkers of disease or ionizing radiation response. In certain embodiments, these small molecules can be detected in biological fluids including but not limited to serum, plasma, lymph, or saliva. In a particularly preferred embodiment, these biomarkers are useful for identifying active (or activated) metabolic pathways following molecular changes predicted by other methods.

The methods of the invention are advantageously used to identify biomarkers for ionizing radiation by functional screening of irradiated cells, including malignant cells. These biomarkers are informative for metabolic and cellular pathways and mechanisms of ionizing radiation response. Importantly, these biomarkers can be used to assist in the evaluation of ionizing radiation response of tumorigenic cells and non-tumorigenic cell types.

Thus, the invention in a further aspect provides cellular products, particularly metabolic products, identified by methods of the invention. These products include preferably products associated with ionizing radiation response and alterations in associated metabolic pathways. Non-limiting examples of metabolic products provided by the invention include phenyl acetate, phenylacetylglycine, 2-phenylacetamide, alpha-N-phenylacetyl-L-glutamine, phenylacetic acid and other metabolites in the phenylalanine pathway, salsolinol, serotonin, butyrylcarnitine, L-Threonine, glucosylgalactosyl hydroxylysine, 1-(9Z, 12Z-octadecadienoyl)-rac-glycerol, 7a-12a-Dihydroxy-3-oxo-4-cholenic acid, or 25:0 N-acyl taurine.

In additional embodiments of this aspect of the invention, these cellular products can be utilized as biomarkers for ionizing radiation exposure.

The invention provides advantageous alternatives to conventional methods for determining tumor response to IR treatment. Current methods require tissue biopsy and immunohistochemical analysis of a patient's tumor. However, repeated biopsy to assess patient response to cancer treatment causes patient discomfort, is costly, and cannot always be performed immediately following IR therapy. The inventive methods using metabolomics, and the biomarkers identified thereby, provide a significant improvement over current methods of tumor analysis. Instead of analyzing a solid tissue sample, cellular products are identified in patient biofluid or serum samples. This type of testing could reduce patient discomfort, permit repeated measurement, and allow more timely assessments.

Specific preferred embodiments of the present invention will be better understood from the following more detailed description of certain preferred embodiments and the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other objects and features of this invention will be better understood from the following detailed description taken in conjunction with the drawings wherein:

FIG. 1 is a depiction of hierarchal clustering of the fold change differences in 13,041 unique masses detected in supernatant or extracellular media from U373 glioma cells and uncultured media (negative control for media background) either treated with 3 Gy of gamma radiation or left untreated. Medium was sampled at three different time points after radiation: one hour, 24 hours, and 48 hours. Samples were measured in triplicates (technical replicates) by liquid chromatography/electrospray ionization mass spectrometry (LC-ESI-TOF-MS). Refer to the figure legend for positive fold changes and negative fold changes. Missing data is solid gray.

FIG. 2 is a color depiction of the hierarchal clustering represented in FIG. 1. Positive fold changes are red, negative fold changes are green, and missing data is grey.

FIG. 3 is a depiction of hierarchal clustering of the fold change differences detected from metabolites of the phenylalanine biochemical pathway detected as described for FIG. 1. Refer to the figure legend for positive fold changes and negative fold changes. Missing data is white.

FIG. 4 is a color depiction of the hierarchal clustering represented in FIG. 3. Positive fold changes are red, negative fold changes are green, and missing data is grey.

FIG. 5 is a schematic diagram of the phenylalanine metabolic pathway in human cells, wherein several metabolites are upregulated as early as one hour following ionizing radiation. Open arrows mark reactions leading to a metabolite with a statistically significant difference at one time point, Horizontally striped dots indicate a metabolite measured in this experiment.

FIG. 6 is a schematic diagram of the experimental design used to measure the metabolic response of glioma cell lines to ionizing radiation. Three different cell lines, U373, U251, and T98G, were treated with 3 Gy of ionizing radiation or mock treatment at two different time points. The cell supernatant was harvested and examined for small molecule metabolites.

FIGS. 7A through 7H are chromatograms from 3 Gy-treated and untreated U373, U251, and T98G cell lines and media only controls. An overlay of chromatograms from all experimental groups demonstrated high reproducibility of LC-ESI-TOF-MS.

FIG. 8 is a Venn diagram of mass features exclusive to glioma cell lines (absent in media). The box represents features that are common to glioma cell lines and the media. Eighty three features were detected at least once in each cell line in the absence of the media while 1428 features were detected in each cell line and each media sample at both time points.

FIGS. 9A through 9F are plots of normalized data of annotated statistically significant molecules from Table 2 showing differences between treatment, control, and media. The bars represent standard error of the mean and n is the number of features measured per factor. FIG. 9A: butyrylcamitine, 24 hours post IR; FIG. 9B: L-Threonine, 1 hour post IR; FIG. 9C: Glucosylgalactosyl hydroxylysine, 24 hours post IR; FIG. 9D: 1-(9Z, 12Z-octadecadienoyl)-rac-glycerol, 24 hours post IR; FIG. 9E: 7a, 12a-Dihydroxy-3-oxo-4-cholenic acid, 24 hours post IR; FIG. 9F: 25:0 N-acyl taurine, 24 hours post IR.

FIGS. 10A through 10C are principal component analysis (PCA) loading plots that display the separation of samples into groups corresponding to cell culture supernatant and media by cell line. The normalized data from masses present in each cell line and condition were used as the input matrix. FIG. 10A is a plot of PCA analysis performed on all secreted molecules. FIG. 10B is a plot of data from the 1 hour time point. FIG. 10C is a plot of data from the 24 hour time point. Open squares correspond to untreated U251 cells, solid squares correspond to 3 Gy treated U251 cells. Open triangles correspond to untreated T98G cells, solid triangles correspond to 3 Gy treated T98G cells. Open circles correspond to untreated U373 cells, solid circles correspond to 3 Gy treated U373 cells, open stars represent untreated media, and closed stars represent 3 Gy treated media.

FIGS. 11A and 11B are depictions of hierarchal clustering of the fold change differences between irradiated and untreated cell culture supernatant by cell line and time. FIG. 11A displays large differences between cell lines and FIG. 11B displays hierarchical clustering of the fold changes between irradiated and untreated cell using cell lines as replicates. Refer to the figure legend for positive fold changes and negative fold changes. Missing data is grey.

FIGS. 12A and 12B are color depictions of the hierarchal clustering represented in FIGS. 11A and 11B. Positive fold changes are red, negative fold changes are green, and missing data is grey.

FIGS. 13A through 13C are Venn Diagrams of secreted mass features having a 2 fold or greater response to IR at 1 hour after treatment (FIG. 13A), 24 hours after treatment (FIG. 13B), and both time points combined (FIG. 13C). These diagrams show the number of secreted features with a common response to IR within and between cell lines.

### DETAILED DESCRIPTION OF PREFERRED EMBODIMENTS

The present invention is more particularly described below and particularly in the Examples set forth herein that are intended as illustrative only since numerous modifications and variations therein will be apparent to those skilled in the art.

As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practitioner regarding the description of the invention.

This invention provides reagents and methods for determining the cellular and/or biochemical effects of ionizing radiation. The term “ionizing radiation” as used herein is intended to encompass high-energy radiation and electromagnetic radiation and includes but is not limited to radiotherapy, x-ray therapy, irradiation, exposure to gamma rays, protons, alpha-particle or beta-particle irradiation, fast neutrons, and ultraviolet. In a preferred embodiment, the result of ionizing radiation administration on cell populations is determined by metabolomics (see Metabolomics, Methods & Protocols, (Wolfram Weckwerth ed., Humana Press 2007).

The term “cellular metabolite” or the plural form, “cellular metabolites,” as used herein refers to any small molecule or mass feature secreted by a cell. In general the size of said metabolites is in the range of about 55 to about 3000 Daltons. A cellular metabolite may include but is not limited to the following: sugars, organic acids, amino acids, fatty acids, and/or hormones. In a preferred embodiment, the cellular metabolite is secreted from cancer cells, particularly glioma cells or melanoma cells.

The phrase “identifying cellular metabolites that are differentially produced” as used herein includes but is not limited to comparisons of cells exposed to ionizing radiation to untreated (control) cells. Detection or measurement of variations in small molecule populations or mass features secreted by a cell, between treated and untreated cells is included in this definition. In a preferred embodiment, alterations in cells or cell activity is measured by determining a profile of changes in small molecules in a treated versus untreated cells. Also included are comparisons between cells treated with different amounts, types or intensities of IR.

Alterations in small molecules such as sugars, organic acids, amino acids, fatty acids, and low molecular weight compounds are measured and used to assess the effects of ionizing radiation on biochemical pathways. The screened small molecules can be involved in a wide range of biological activities including, but not limited to inflammation, anti-inflammation, vasodilation, neuroprotection, fatty acid metabolism, products of collagen matrix degradation, oxidative stress, antioxidant activity, DNA replication and cell cycle control, methylation, biosynthesis of nucleotides, carbohydrates, amino acids and lipids, among others. Small molecule metabolites are precursors, intermediates and/or end products of biochemical reactions in vivo. Alterations in specific subsets of molecules can correspond to a particular biochemical pathway and thus reveal the biochemical effects of ionizing radiation. In a particularly preferred embodiment, metabolomics is used to examine the effects of IR on cancer cells.

Glioma cells are generally derived from glial cell tumors and in particular brain tumors. However, gliomas may develop in the spinal cord or any other part of the central nervous system. In a preferred embodiment, the methods described herein specify “glioma,” but methods are not to be limited solely to glioma tumors. In additional embodiments, disclosed methods include “glioblastoma multiforme” (GBM) brain tumors, the most common type of brain tumor, as well as non-CNS tumors including melanomas as one example.

In preferred embodiments the methods of the present invention are used to assess differential cellular metabolite content and production from malignant or tumorigenic tissue. The term “tumor” or “malignant” includes cancerous tissue at any of the conventional four cancer stages (I-IV) as well as precancerous tissue. In alternative embodiments, the methods of the invention may examine precancerous tissue. The term “precancerous” includes a stage of abnormal tissue growth that is likely or predisposed to develop into a malignant tumor.

The term “physical separation method” as used herein refers to any method known to those with skill in the art sufficient to produce a profile of changes and differences in small molecules produced by cells, including tumor cells, exposed to ionizing radiation according to the methods of this invention. In a preferred embodiment, physical separation methods permit detection of small molecules including but not limited to sugars, organic acids, amino acids, fatty acids and low molecular weight compounds. Advantageous methods for separation comprise chromatography, most preferably liquid chromatography (LC), and identification methods comprise mass spectrometry techniques. In particular embodiments, this analysis is performed by liquid chromatography/electrospray ionization mass spectrometry (LC-ESI-TOF-MS), however it will be understood that small molecules as set forth herein can be detected using alternative spectrometry methods or other methods known in the art. Similar analyses have been applied to other biological systems in the art (Want et al, 2005, Chem Bio Chem. 6:1941-51), providing biomarkers of disease or toxic responses that can be detected in biological fluids (Sabatine et al, 2005, Circulation 112:3868-875).

The term “biomarker” as used herein refers, inter alia to small molecules that exhibit significant alterations between treated and untreated controls, particularly with regard to IR treatment. In preferred embodiments, biomarkers are identified as set forth above, by methods including LC-ESI-TOF-MS.

In preferred embodiments, the following small molecules are provided herein, taken alone or in any informative combination, as biomarkers of cancer cell response to ionizing radiation: phenylacetate, phenylacetylglycine, 2-phenylacetamide, alpha-N-phenylacetyl-L-glutamine, phenylacetic acid and other metabolites in the phenylalanine pathway, salsolinol, serotonin, butyrylcarnitine, L-threonine, glucosylgalactosyl hydroxylysine, 1-(9Z,12Z-octadecadienoyl)-rac-glycerol, 7a-12a-dihydroxy-3-oxo-4-cholenic acid, or 25:0 N-acyl taurine.

The measurement of these biomarkers in patient blood, plasma, sera, lymph, saliva, urine, or other patient specimen can provide a diagnostic or prognostic assessment of a patient's response to IR.

The term “biomarker profile” as used herein refers to a plurality of biomarkers identified by the inventive methods. Biomarker profiles according to the invention can provide a molecular “fingerprint” of the effects of ionizing radiation and identify small molecules significantly altered following ionizing radiation exposure. In preferred embodiments, biomarker profiles can be used to diagnose radiation exposure or cellular response to radiation treatment.

In a further embodiment, the diagnosis of radiation exposure is not limited to medical exposure, and may further include, but is not limited to the following examples: accidental radiation exposure, war-related, or bioterror radiation exposure. The phrase “outside of medical treatment” includes the above-mentioned non-limiting examples.

A “biological sample” includes but is not limited to cells cultured in vitro, a patient sample, or biopsied cells dispersed and cultured in vitro. A “patient” may be a human or animal. A “patient sample” includes but is not limited to blood, plasma, serum, lymph, urine, cerebrospinal fluid, saliva or any other biofluid or waste.

### EXAMPLES

The Examples which follow are illustrative of specific embodiments of the invention, and various uses thereof. They set forth for explanatory purposes only, and are not to be taken as limiting the invention.

All references cited herein are incorporated by reference. U.S. Publication No. 20070248947A1 of Oct. 25, 2007 and PCT Publication No. WO 2007/120699 of Oct. 25, 2007 are explicitly herein incorporated by reference.

### Example 1

### Metabolomic Analysis of U373 Glioma Cells Treated with Ionizing Radiation

U373 glioma cells were exposed to a conventional dose of ionizing radiation to demonstrate that metabolomics was useful for examining cellular response to IR and to identify biomarkers for response. The treated cells were analyzed as set forth below to determine changes in a total dynamic set of small molecules present in cells according to health and disease or insult states. Small molecules such as sugars, organic acids, amino acids, fatty acids and signaling low molecular weight compounds were understood to participate in and reveal functional mechanisms of cellular response to pathological or radiation insult. These analyses were also used to identify active pathways following molecular changes implicated by other methods including for example transcriptomics and proteomics.

U373 glioma cells and uncultured media (negative control for media background) were either treated with 3 Gy of gamma radiation or left untreated. This dose of IR represents the standard daily dose delivered in the treatment of glioblastoma multiforme (GBM) during conventional fractionated treatment. The media were sampled at three different time points following radiation exposure: 1 (one) hour, 24 hours, and 48 hours. Medium collected from radiation exposed cells and control (“no-treatment”) cells was subjected to liquid chromatography and electrospray ionization mass spectrometry (LC-ESI-TOF-MS) to assess changes and differences in the metabolome produced by the cells in the presence and absence of ionizing radiation exposure. Samples were measured in triplicate (technical replicates) by LC-ESI-TOF-MS. Analysis of the standard deviation of the retention times revealed that the majority of masses were comparable across LC-ESI-TOF-MS runs with the majority of replicate measurements detected within 20 seconds of each other. This finding demonstrates the reproducibility of the detection method used.

Each sample had three replicates injected into a 2.1×200 mm HPLC C18 column run on a 120 minute gradient from 5% acetonitrile, 95% water, 0.1% formic acid to 100% acetonitrile, 0.1% formic acid at a flow rate of 40 L/min. The flow through was introduced into an Agilent 1100 series LC-ESI-TOF-MS. Data was collected from 0-1500 m/z range throughout the run. The raw data was loaded into the Analyst QS program (Agilent) to visualize retention time and mass features prior to data analysis. Mass Hunter MF (Agilent) software was used to deconvolute the data and determine the abundance of each mass. Masses within the range of 80-1500 m/z, a charge of +1, and at least 2 ions were included in this analysis. A mass was considered to be the same across LC-ESI-TOF-MS runs using a simple binning algorithm based on mass and retention time. Bins were created when masses differed by 10 ppm or if the same mass had a retention time difference greater than one minute. Significance tests were determined by performing ANOVAs on the log base 2 transformed abundance values. A complete randomized design was used with the following formula:

{Log₂(abundance_{tb})=treatmentₜ+glioma line_{b}+error_{tb}}

on each bin to determine significance. Imputation was not performed and missing data was omitted from the data analysis affecting the degrees of freedom for each test of significance. Clustering of the fold changes was performed using Cluster 3.0 software (De Hoon et al., 2004, Bioinformatics 20(Suppl. 1):i101-i108). Principal component analysis was performed using the open source statistical package R and the pcaMethods library. Significant differences in secreted metabolites were detected between irradiated and untreated glioma cell lines. Statistically significant differential secreted metabolites were determined using an ANOVA model. The distribution of abundances appears to follow a normal distribution. Molecules with a p-value less than 0.05 and more than 3 degrees of freedom were considered significant when comparing individual cell lines.

The plurality of small molecules identified using these methods was then annotated by comparison with exact neutral masses of chemicals catalogued in public databases, e.g., METLIN Metabolite Database, Human Metabolome Database (HMDB), Kyoto Encyclopedia of Genes and Genomes (KEGG), and the Biological Magnetic Resonance Data Bank (BMRB). Mass spectrometry analysis also included predicted chemical structures of small molecules based upon exact mass, although currently-available public databases do not in every instance include matching small molecules due to the lack of complete databases with the full spectrum of human metabolites.

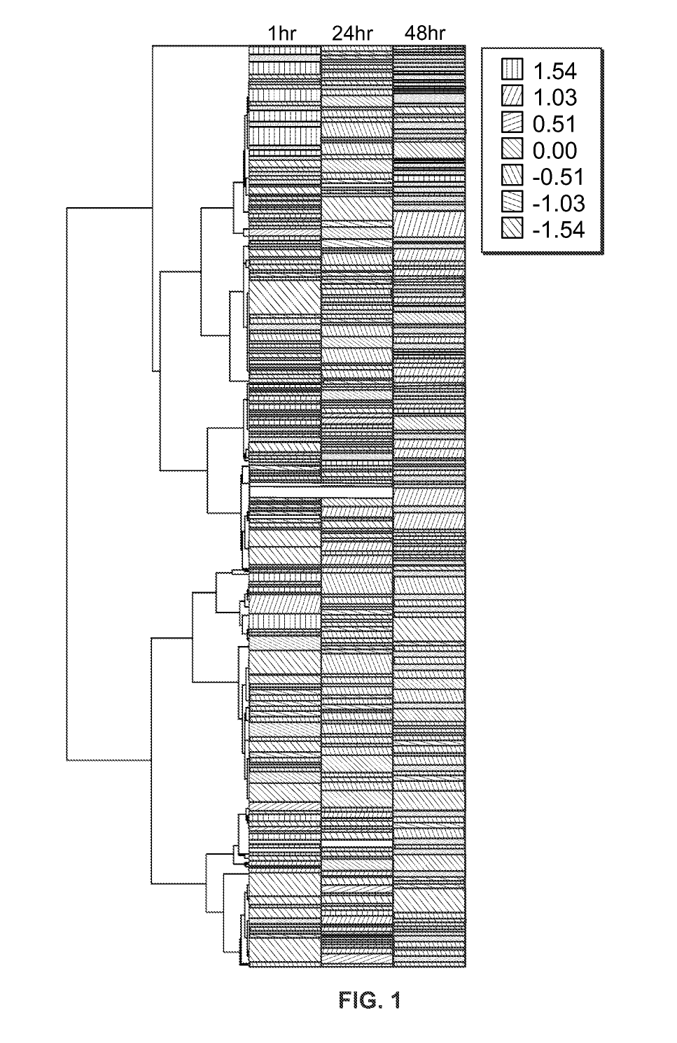

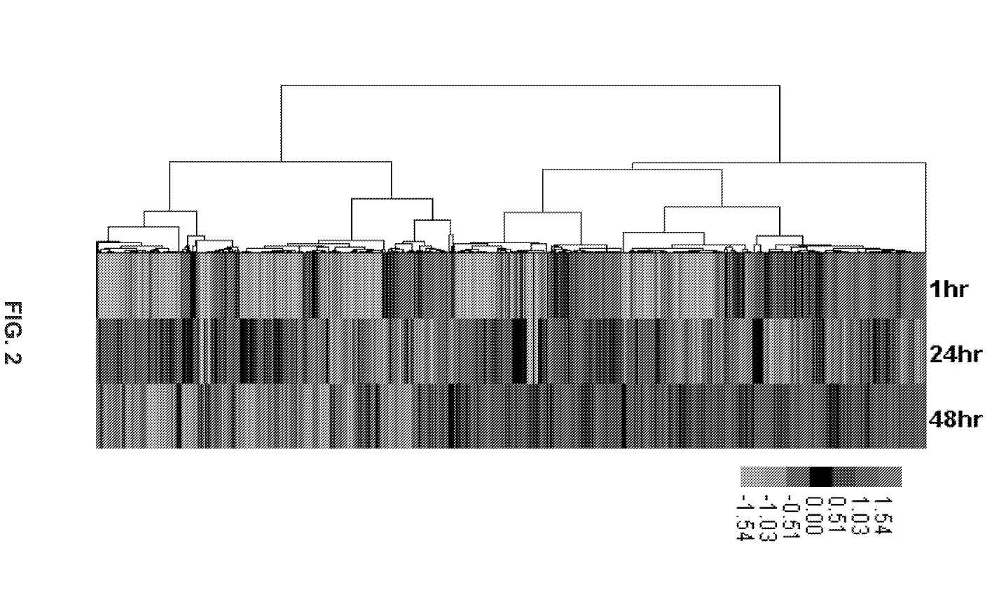

A total of 161,923 mass signatures were detected by LC-ESI-TOF-MS representing 48,608 unique neutral masses. Standard adducts from sodium and potassium were removed from the spectra, which was also subject to deisotoping. This large number of neutral masses were measured because the data contained signals that were measured one or two times across the entire experiment. Masses measured 2 or fewer times were considered to be spurious and removed from the data set. The final data set used for analysis contained 13,041 masses (˜27% of the unique masses, FIG. 1 and FIG. 2). 3,356 (26%) of these masses corresponded to small molecules detected only in glioma samples. 471 of these masses were present only in the irradiated cells and 202 masses were measured only in the untreated glioma samples.

### Example 2

### Phenylalanine and Other Metabolites: Biomarkers of Ionizing Radiation in U373 Glioma Cells

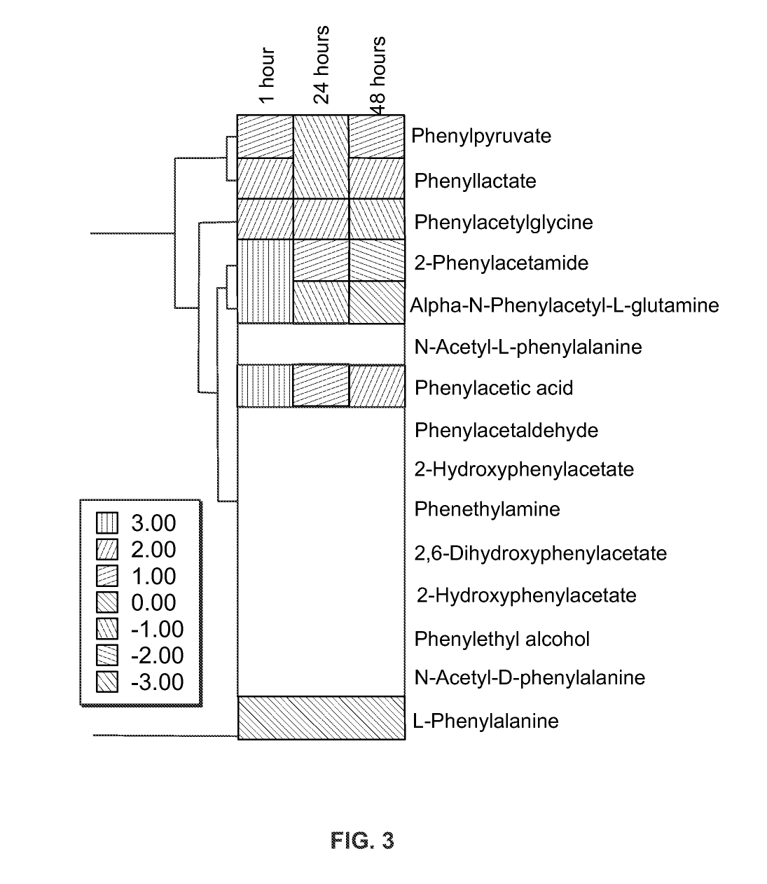

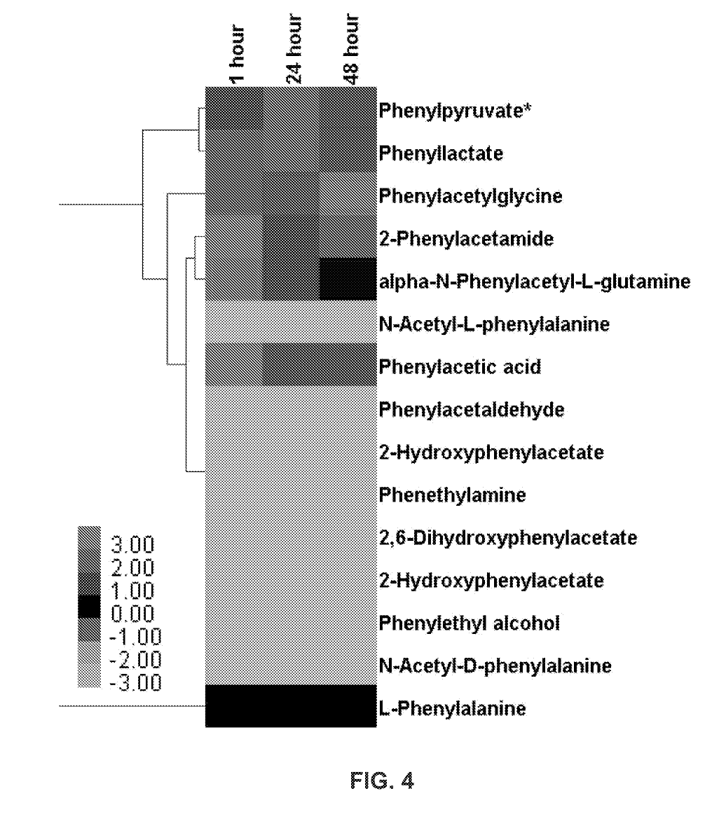

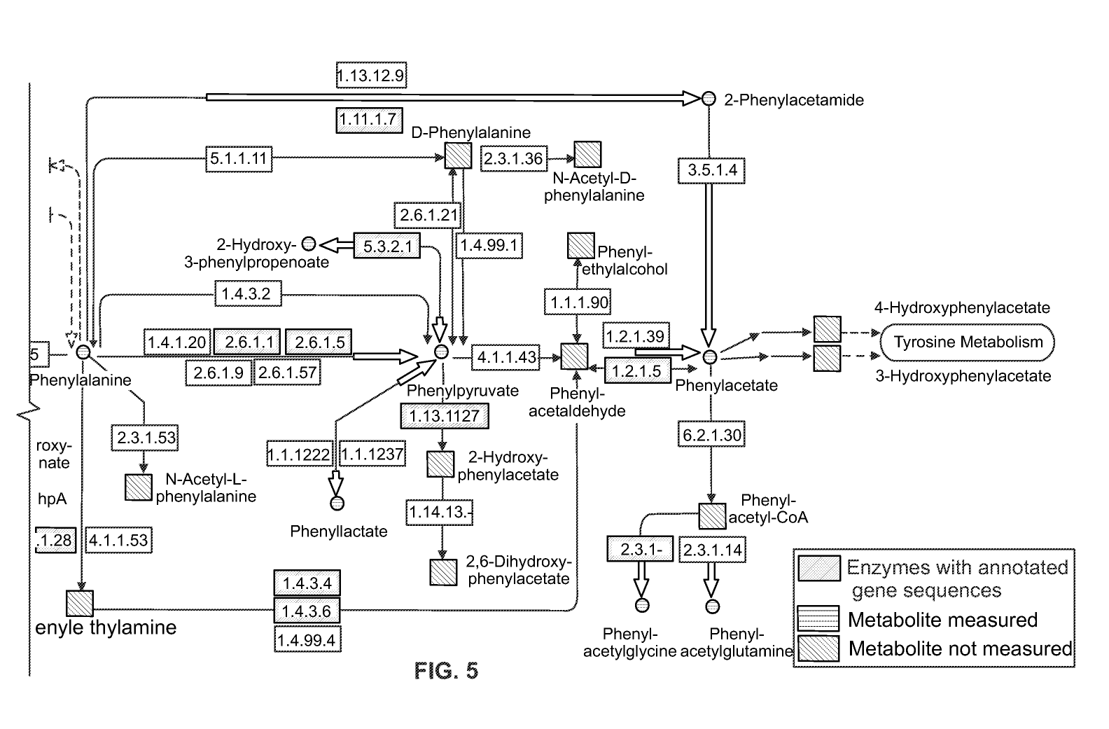

The results of the biomarker identification experiments disclosed in Example 1 were analyzed to identify particular metabolites and metabolic pathways showing differential activity in irradiated and control (nonirradiated) samples. One biochemical pathway, the phenylalanine pathway, was particularly significant at one hour after irradiation. Several metabolites present in phenylalanine metabolism had significant changes after IR. These changes are shown in FIG. 3 and FIG. 4, which illustrates the fold-change differences detected from metabolites of the phenylalanine biochemical pathway. Refer to the legend for designations representing positive and negative fold changes. Missing data is solid white. A schematic diagram of the phenylalanine metabolic pathway is shown in FIG. 5. Open arrows mark reactions leading to a metabolite with a statistically significant difference at one time point, dots indicate a metabolite measured in this experiment, striped boxes represent a metabolite that was not measured in this experiment. One putative metabolite in the phenylalanine metabolism pathway, phenylacetate, was 3.5 fold more prevalent 1 hour after ionizing radiation (p=0.002) than in non-irradiated samples.

Phenylacetate (PA) is a naturally occurring metabolite present in the phenylalanine metabolic pathway that is typically detected in serum. (see FIG. 5). Previous research has demonstrated that PA can inhibit the growth of tumor cells in vitro and in vivo (Samid et al., 1994, Cancer Res. 54:891-5). It has been suggested that PA may actually potentiate the response of tumor cells to IR (Miller et al., 1997, Int J Radiat Biol. 72:211-8.). Further investigations found that the amount of PA required to affect tumor growth is cell line dependent and that brain tumors are more sensitive to its effects than other tumor lines, but these results also called into question radiopotentiation of PA (Ozawa et al., 1999, Cancer Lett. 142:139-46). A phase II clinical study of PA did not find a significant response in patients with GBM (Chang et al., 1999, J Clin Oncol. 17:984-90). Interestingly, metabolites in the phenylalanine pathway feed into the production of DOPAchrome that leads to the production of DHICA which is known to increase radioresistance in skin cancer (FIG. 5).

The discovery of this pathway using unbiased methods demonstrates the power of metabolomics to identify metabolic pathways that respond to IR. Other metabolites in this pathway, which are included as biomarkers in this invention and were detected following exposure of two additional glioma cell lines (T98G and U251) to 3 Gy of ionizing radiation are: phenylpyruvate, phenylacetylglycine, 1-phenylacetamide, alpha-N-phenylacetyl-L-glutamine and phenylacetic acid, which where all significantly upregulated in response to IR. In addition, enol-phenylpyruvate, phenylacetaldehyde, L-adrenaline, L-noradrenaline, and 3,4-dihydroxymandelate were altered following IR. Altogether, these findings converge towards two main metabolic pathways: phenylalanine and tyrosine, and indicate that changes in these metabolites comprise biomarkers for IR.

Other molecules that can serve as biomarkers of IR in cancer are: salsolinol (60% decrease at 24 hours, p<0.0001) and serotonin (7-fold increase at 1 hours, p<0.0001; 60% decrease at 24 hours, p<0.018; 2.5 increase at 48 hours, p=0.005). Interestingly salsolinol, a derivate of dopamine, is a neurotoxin that induces apoptosis in dopaminergic neurons (Mravec, 2006, Physiol Res. 55:353-64). Salsolinol is significantly decreased at 24 hours after irradiation. Serotonin accumulation was significantly increased at 1 hour and 48 hours, but decreased at 24 hours after irradiation. Serotonin has been shown to cause an increase in IL-6 release in glioma cell lines (Lieb et al., 2005, J Neurochem. 93:549-59).

In addition, the following metabolites were also altered at statistically significantly levels in response to IR: 2,7-Anhydro-alpha-N-acetylneuraminic acid or 2-Deoxy-2,3-dehydro-N-acetylneuraminic acid, which was 2.5-fold upregulated at 48 hours, p=0.023; and 10-fold upregulated at 1 hour, p=0.008; N-Acetylneuraminate which was 1.4 fold downregulated at 48 hours, p=0.005; N-Acetyl-O-acetylneuraminate, increased 1.2-fold at 48 hours, p=0.016; Indoxyl sulfate or 4-Phospho-L-aspartate, 3.4-fold downregulated at 24 hours, p=0.03; 5.9-fold downregulated at 1 hour, p=0.007; N-Acetyl-L-histidine 1.28-fold downregulated at 1 hour, p=0.003; 1.4-fold upregulated at 48 hours, p=0.0001; Isopentenyladenine or L-Acetylcarnitine up 4.8-fold at 1 hour, p=0.01; increased 2.9-fold at 48 hours, p=0.0068; and reticuline, which was upregulated 4.1-fold at 1 hour, p=0.007. Overall, these results reflect that in vitro metabolomics of glioma cells is a robust alternative for the detection of small molecules, which can serve as translational biomarkers of ionizing radiation response.

### Example 3

### Metabolomic Analysis of Multiple Glioma Cell Lines Exposed to Ionizing Radiation

Secreted or excreted small molecule metabolites from malignant glioma cell lines in response to IR were evaluated using metabolomics. Glioma cell lines were treated with 3 Gy of IR and response was analyzed by metabolite profiling of secreted small molecules using liquid chromatography electrospray ionization time of flight mass spectroscopy (LC-ESI-TOF-MS). Statistically significant differences in the abundance of putative secreted metabolites were detected between irradiated and untreated cell lines.

U373, T98G, and U251 GBM cell lines were exposed to a conventional dose (3 Gy) of ionizing radiation to identify biomarkers for cellular response to IR. The treated cells were analyzed as set forth below to determine changes in a total dynamic set of small molecules present in cells according to health and disease or insult states. These experiments were performed generally as set forth in Example 1, however in the present Example, three glioblastoma cell lines were examined in an effort to provide a more robust analysis and to identify common metabolites among glioma cell lines. Because metabolic changes are inherent to cancer pathogenesis (Griffin & Shockcor, 2004, Nat Rev Cancer 4: 551-61; Jensen, 2006, Neurosurg Focus 20: E24; Brown & Wilson, 2004, Nat Rev Cancer 4: 437-47; Yetkin et al., 2002, Neuroimaging Clin N Am 12: 537-52) they can also be directly involved in tumor response to ionizing radiation. This study examined whether there were common metabolic changes to different glioma cell lines in response to IR.

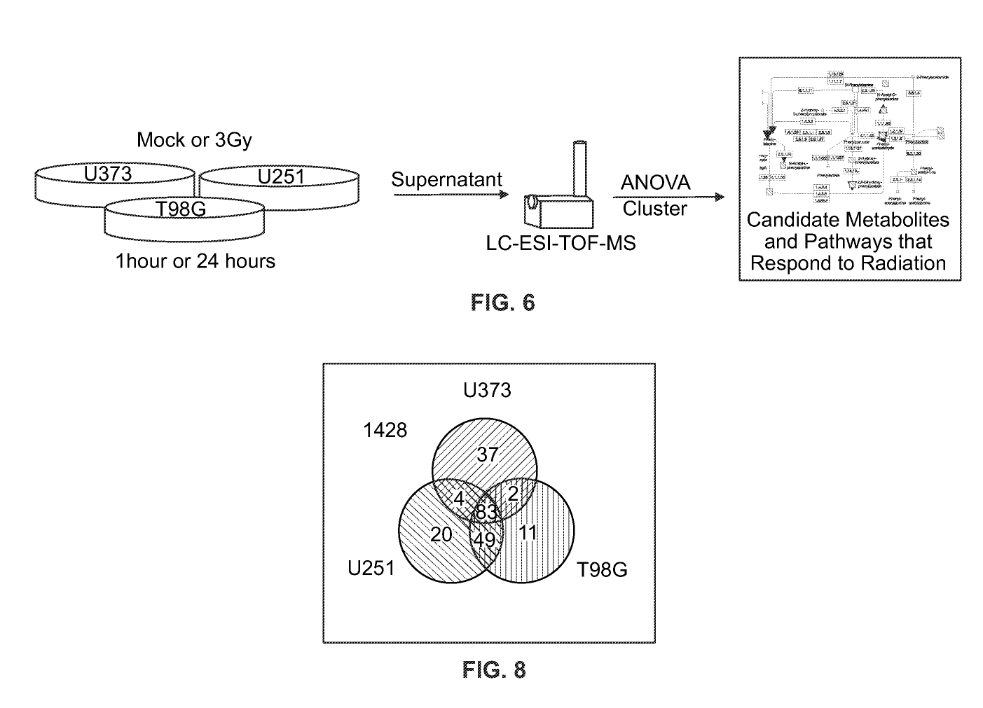

The GBM cells lines U373 (ATCC# HTB 17), T98G (ATCC #CRL-1690), and U251 (provided by Paul Harari, University of Wisconsin-Madison) were cultured under standard conditions to 50-70% confluence and then exposed to 3 Gy of ionizing radiation (IR) or placed in the irradiator but not exposed to the source (mock treatment) (FIG. 6). This dose of IR is comparable to the daily dose delivered in the treatment of GBM during fractionated radiotherapy. The cell cultures were sampled at one and 24 hours following IR. Media without cells were treated in the same manner as cell cultures and used as a reference to detect cell specific metabolites except for line U373 where no irradiated media was collected. Only one medium sample was used as the untreated control for U251 and T98G. These uncultured, untreated medium samples were duplicated in the data analysis and represented both 1 and 24 hour time points for the untreated media measurements.

Cell culture media supernatant from the irradiated and untreated glioma cell lines was collected at one and 24 hours post-IR and stored at −80° C. The samples were simultaneously thawed and 125 μL was processed for liquid chromatography using Millipore 3 kDa Centricon regenerated cellulose columns (Millipore) to remove proteins and large molecular weight biomolecules. The flowthrough was retained for analysis as it contains small molecules free of high molecular weight compounds. The flowthrough was lyophilized and suspended in 50 μL 0.1% formic acid.

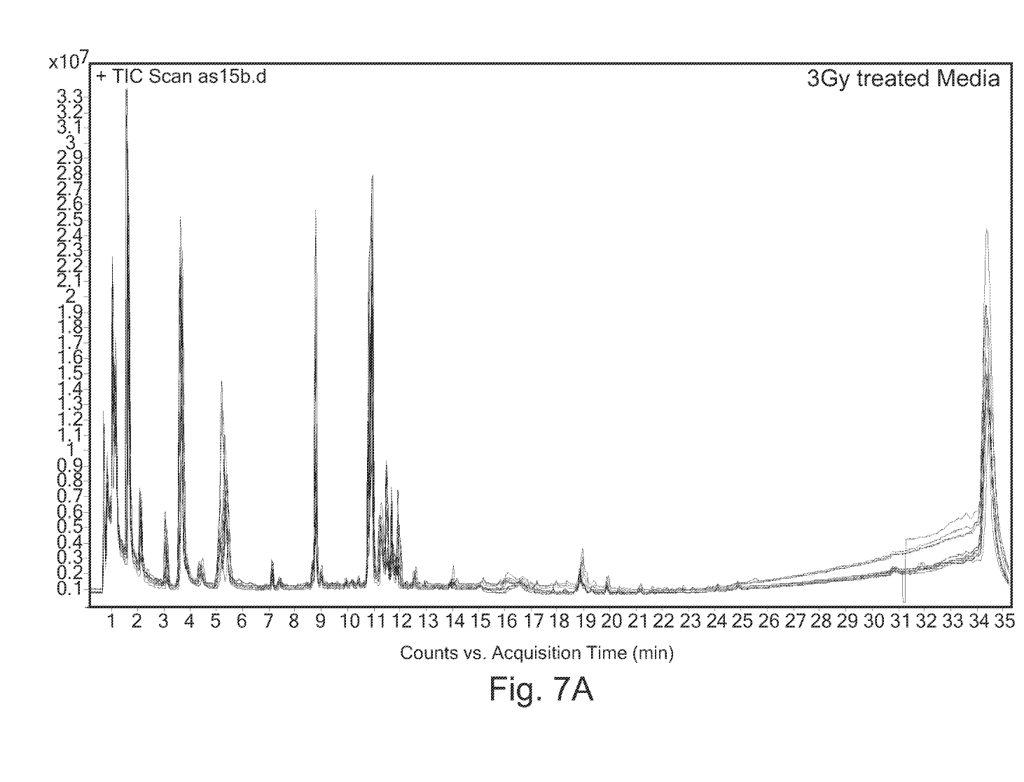

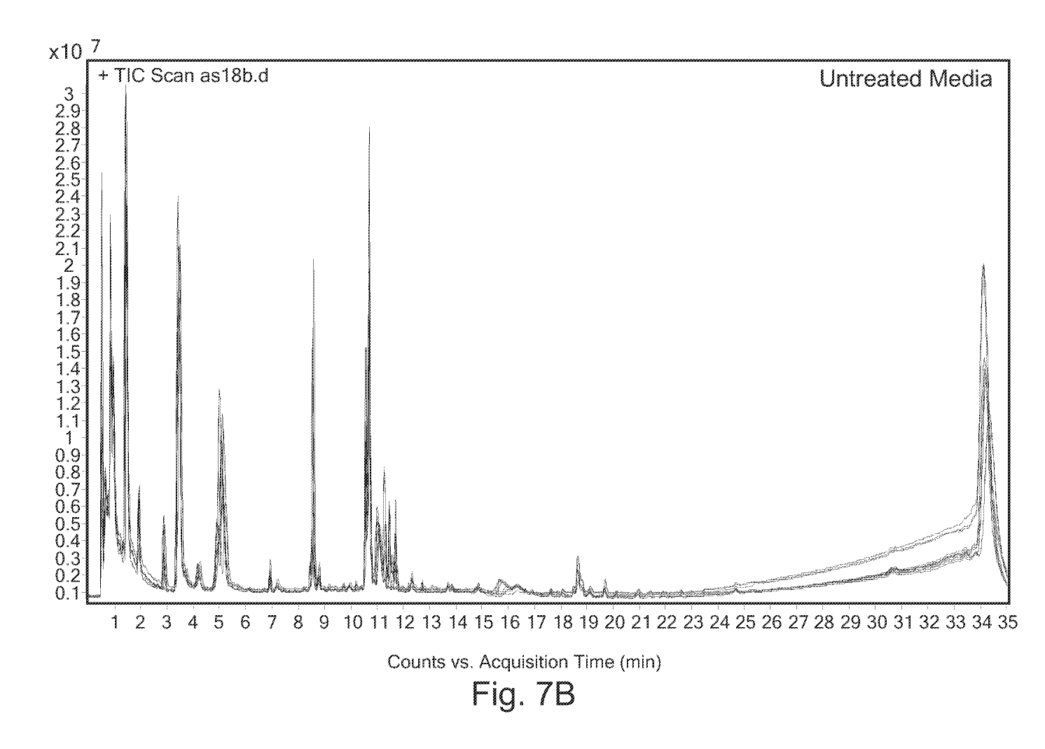

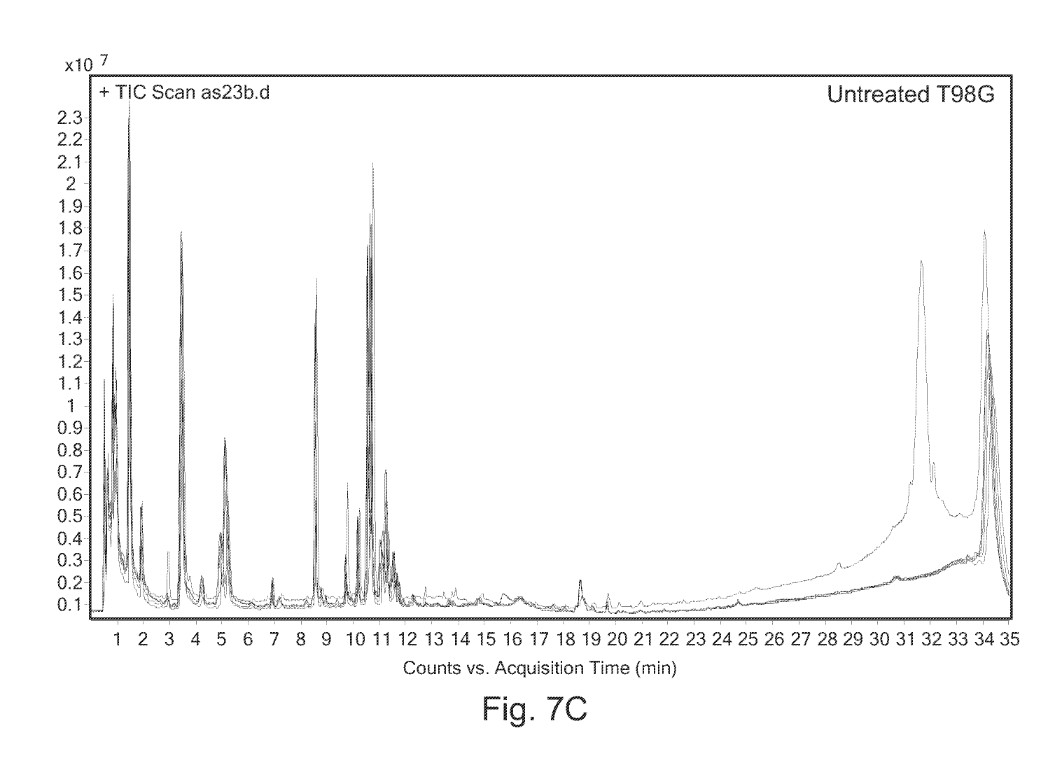

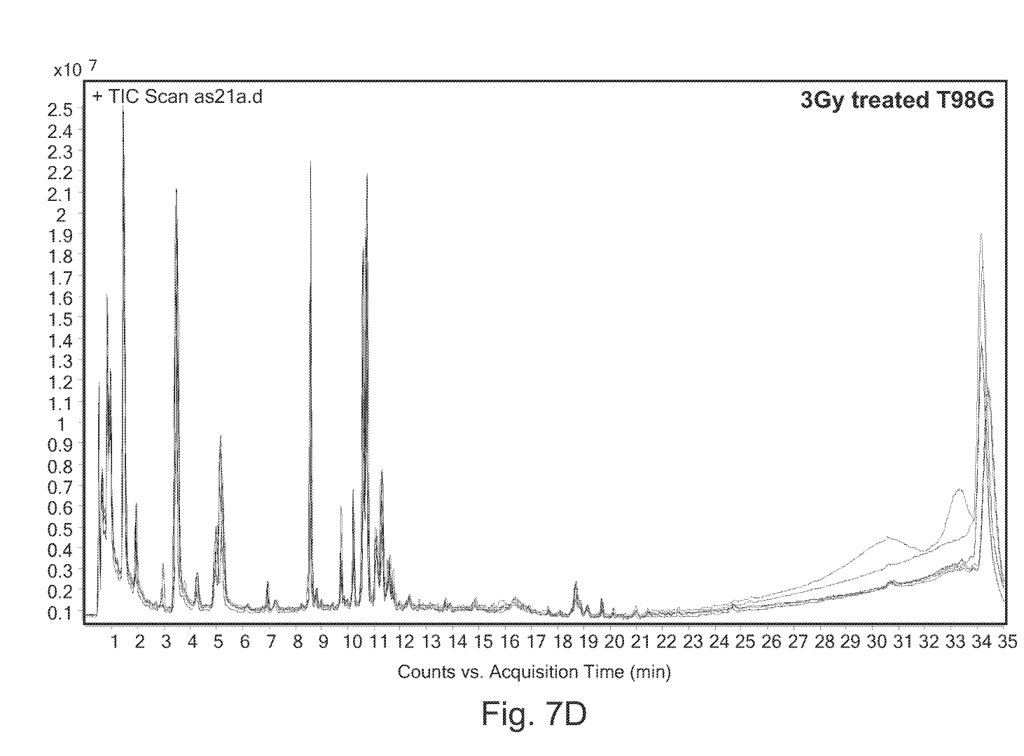

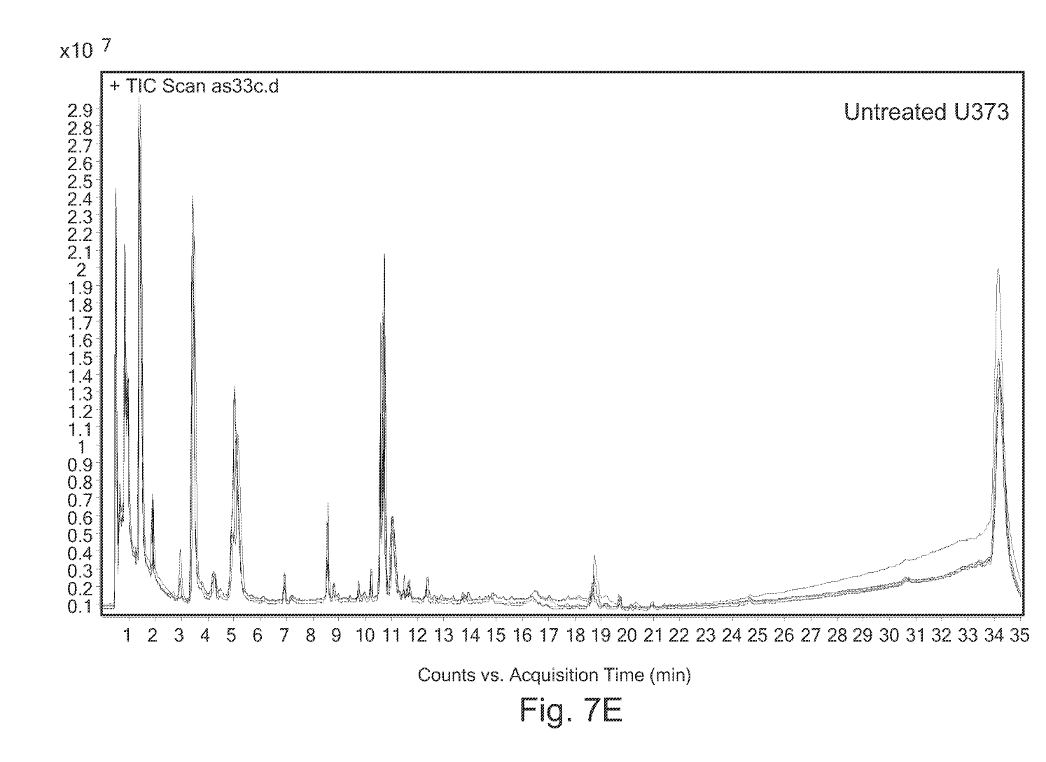

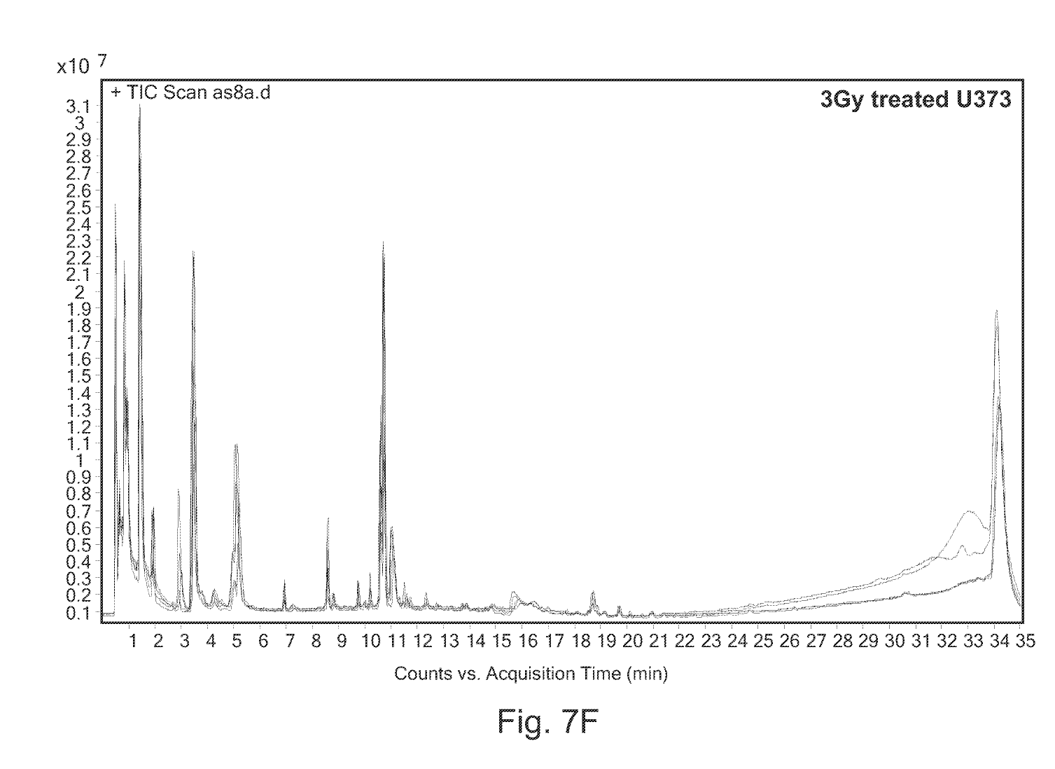

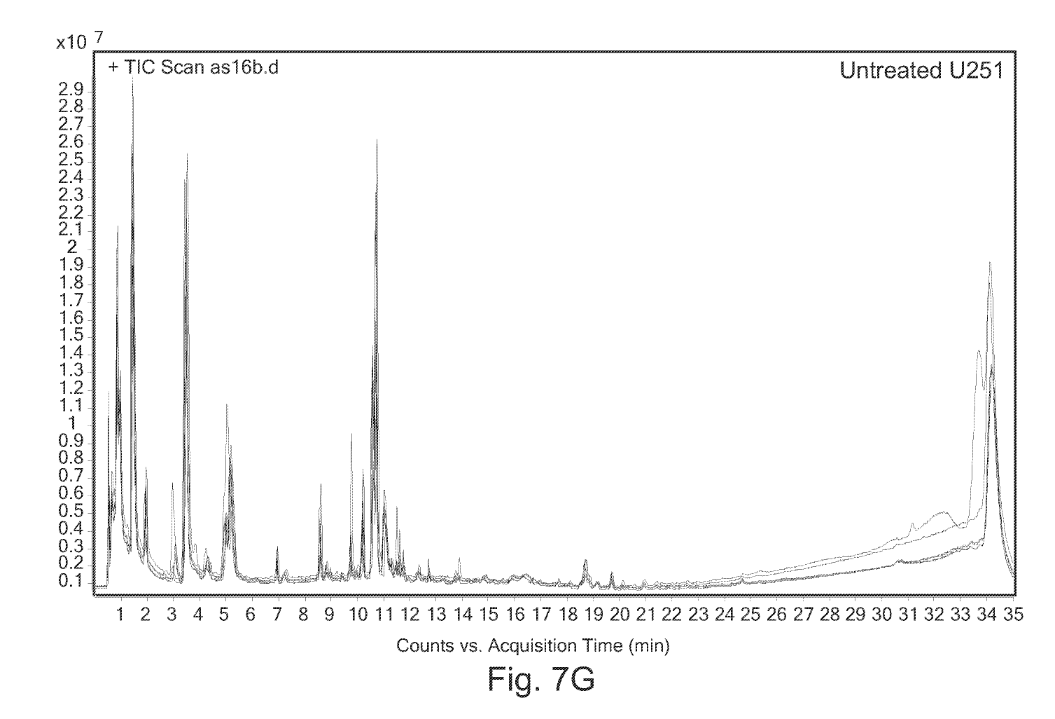

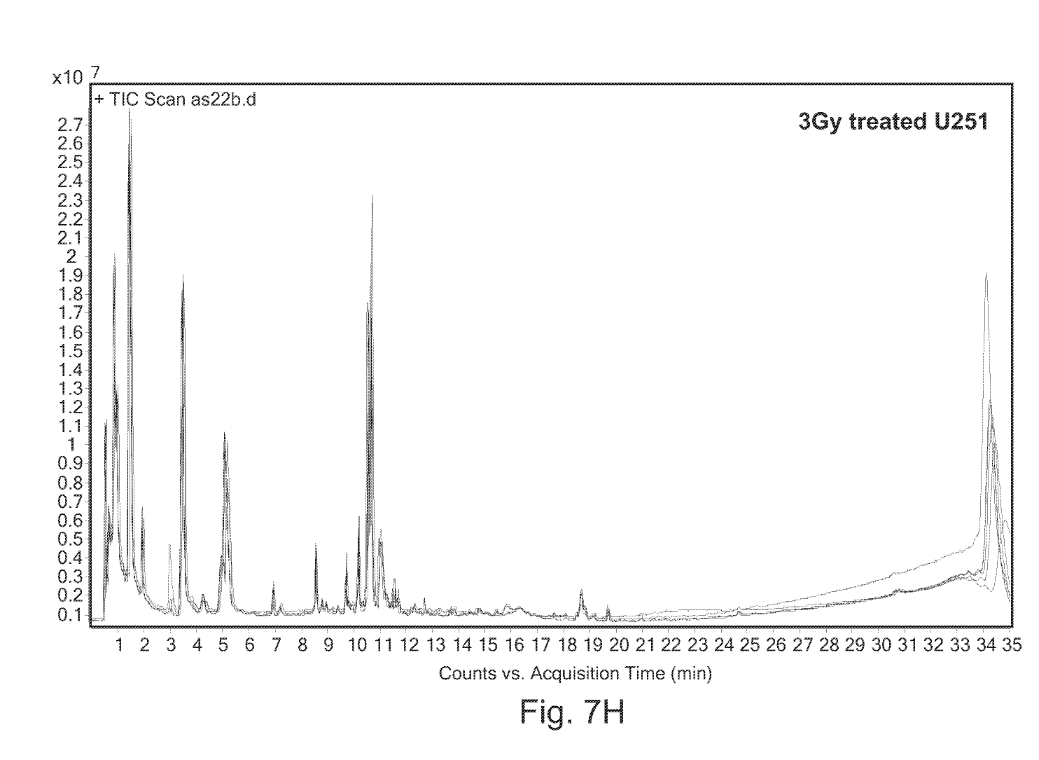

5 μL of each sample was injected in triplicate into a 2.1×30 mm Zorbax C18-SB column run on a 30 minute gradient from 5% acetonitrile, 95% water, 0.1% formic acid to 100% acetonitrile, 0.1% formic acid at a flow rate of 200 μL/min. The flowthrough was introduced into an Agilent 1100 series LC-ESI-TOF-MS. Data were collected from 50-1500 m/z range throughout the run. The settings for the ion source were: gas temperature 350 C, drying gas 9.5 L/min, nebulizer 30 psig, capillary 4000 V. The settings for the TOF were: fragmentor 185 V, Skimmer 60 V, OCT RF 250 V. The chromatograms were inspected after each LC-MS run and any samples with abnormal chromatography were repeated. Data were extracted from the chromatographs using all information from 0-27 minutes (FIG. 7).

The Mass Hunter MFE version 44 software (Agilent) was used to deconvolute the data, which consists of removing isotopes and adducts, and establishing the abundance of each mass feature. The abundance was calculated as the sum of the isotopic and adducts peaks folded into a single mass feature. Masses measured within the range of 50-1500 m/z, m/z charge of +1, a minimum abundance greater than 0.001%, a signal to noise value greater than or equal to 5. After data deconvolution mass features with at least two ions and an abundance value greater than 0.05 quantile were included in the data set used for binning. A set of mass features was considered to be the same across LC-ESI-MS-TOF runs using a simple binning algorithm based on mass and retention time. Mass features under 175 Da were binned by 0.00001×mass, while those from 176 Da-300 Da were binned by 0.000007×mass and 0.000005×mass when over 300 Da with a retention time difference of less than seven seconds. The binning process was used to create unique compound identities (cpdID) representing a single small molecule.

These binned data were separated into two distinct sets serving different purposes. One set was used for qualitative analysis and the other data set was used for statistical analysis. The data set used for qualitative analysis contained all mass bins that contained at least 3 masses in order to remove compound IDs that may be due to experimental artifacts such as rare fragments or spurious integration of background peaks. The second data set was used for statistical analysis and contained mass bins that were detected in each cell line and treatment at a given time point. The mass feature bins (cpdIDs) used for statistical analysis were also filtered against the media and cpdIDs with an average abundance less than or equal to the media were removed from interpretation because they may not represent secreted/excreted metabolites.

Prior to statistical analysis the data was divided into two subsets and statistical analysis was performed on the putative secreted mass feature subset. Significance tests were determined by performing ANOVAs on the log base 2 transformed row and column median normalized abundance values. A randomized complete block design was used with the following formula Log₂(abundance_{tb})=treatmentₜ+glioma line_{b}+error_{tb} on each mass feature bin to determine significance. This model was only used on mass features that were measured in each cell line and treatment. Imputation was not performed and missing data were omitted from the data analysis affecting the degrees of freedom for each test of significance. Principal component analysis (PCA) was performed using the open source statistical package R and the pcaMethods library.

The average neutral exact mass of each mass feature bin was queried against the public searchable databases METLIN (http://metlin.scripps.edu), The Human Metabolome Database (http://www.hmdb.ca), Kyoto Encyclopedia of Genes and Genomes (www.genomejp/kegg/), and the Biological Magnetic Resonance Bank (http://www.bmrb.wisc.edu/metabolomics/) for candidate identities. Measured mass features were considered to match a small molecule present in the databases if their exact masses were within 10 parts per million of the annotated database molecule (0.00001×mass).

The small molecules altered in response to IR were a diverse group of metabolites involved in fatty acid metabolism, products of collagen matrix degradation, and other cellular processes, as set forth in greater detail in Example 4 below. As radiotherapy remains the primary treatment modality for malignant glioma and prognosis remains poor, defining the metabolic or biochemical response of gliomas to IR provides insight into cellular processes contributing towards their intrinsic radiation resistance. In addition, these molecules could also serve as candidate biomarkers to predict the response and/or resistance of IR in malignant gliomas.

### Example 4

### Biomarkers of Ionizing Radiation in Glioma Cell Lines

The results of the metabolite identification experiments disclosed in Example 3 were used to identify particular metabolites and metabolic pathways showing differential activity in irradiated and control (nonirradiated) samples. The data set used for qualitative analysis contained all mass bins with at least three masses in order to remove compound IDs that may be due to experimental artifacts such as rare fragments or spurious integration of background peaks. This data set contained 13,308 mass feature bins and was used for metabolite annotation and analysis of compounds that were present only in one condition or specific cell line. The second data set, subject to statistical analysis, contained mass bins that were detected in each cell line and treatment and detected above the media background abundance at a given time point. This second data set was used to determine statistical significance of differentially accumulated small molecules in response to IR. Using these criteria, statistical analysis was performed on a total of 1339 mass feature bins.

Analysis of the “metabolic profiles” of each glioma cell line was performed using the qualitative data set (FIG. 8). The data were examined for metabolites or mass features not found in the medium but unique to one cell line or common metabolites among cell lines. 206 metabolites or mass features were exclusively detected in the supernatant of cell cultures and absent in cell culture media. U373 (18%) had the most unique secreted metabolites or mass features followed by U251 (10%) and T98G (5%). 40% of uniquely secreted or excreted metabolites or mass features were common to all three cell lines (FIG. 8), thus when the effect of the media is removed from the analysis, it is likely that they share a similar signature or complement of secreted metabolites and mass features.

Ionizing radiation induces statistically significant changes to small molecule metabolites in glioblastoma cell lines. Statistically significant differences in the abundance of secreted small molecule metabolites were detected between irradiated and untreated glioma cell lines. These differences were determined by an ANOVA model that used the different cell lines as biological replicates. In the ANOVA model, a p-value <0.025 was used to determine significance. Among the secreted features, 125 small molecules (9%) were significantly different; with 4 (0.2%) common to both time points and 55 (4.1%) and 73 (5.5%) small molecules detected at one and 24 hour time points, respectively. The majority of differentially-secreted mass features (77%) did not have putative metabolite annotations in public small molecule metabolite databases. (Table 1) This finding is a reflection of the relatively early status of metabolomics in comparison to other “omics” and it also highlights a heightened demand for population of metabolomic databases.

TABLE 1
Statistically significant differentially secreted mass features and putative
metabolites from three GBM cell lines after 3GY IR treatment.

| | Monoisotopic | Retention | | Fold | P Value | Putative Human |
|---|---|---|---|---|---|---|
| cpdID | Mass | Time | Time | Change | alpha = 0.025 | Metabolite |
| 577 | 92.0595 | 1.433283 | 1 h | 1.54 | 0.0016 | no hit |
| 687 | 97.0894 | 4.234614 | 1 h | −1.99 | 0.0078 | no hit |
| 1181 | 113.084 | 4.814683 | 1 h | −2.74 | 0.0093 | no hit |
| 1323 | 115.0628 | 10.21004 | 24 h | 2.82 | 0.0013 | Proline |
| 1852 | 129.1519 | 4.810348 | 24 h | 1.95 | 0.0056 | no hit |
| 2293 | 143.0943 | 3.493017 | 1 h | −1.70 | 0.0073 | Proline betaine |
| 2515 | 148.049 | 1.436515 | 24 h | 1.25 | 0.0185 | L- |
| | | | | | | Aspartylhydroxamate |
| | | | | | | (oxidation |
| | | | | | | product) |
| 2628 | 150.9764 | 9.751492 | 24 h | 2.81 | 0.0228 | no hit |
| 2629 | 150.9764 | 10.21161 | 24 h | 1.43 | 0.0100 | no hit |
| 3129 | 162.0666 | 23.84309 | 24 h | −1.94 | 0.0170 | no hit |
| 3143 | 162.1041 | 14.83821 | 1 h | 1.98 | 0.0064 | no hit |
| 3788 | 177.9856 | 0.708176 | 1 h | 1.46 | 0.0000 | no hit |
| 3856 | 179.0897 | 1.623656 | 24 h | −1.66 | 0.0005 | no hit |
| 3955 | 181.943 | 2.840092 | 24 h | −2.39 | 0.0137 | no hit |
| 5611 | 213.1289 | 2.008389 | 1 h | −1.67 | 0.0049 | no hit |
| 5612 | 213.1334 | 2.55298 | 1 h | −1.84 | 0.0038 | no hit |
| 5634 | 213.2453 | 17.33491 | 1 h | 3.09 | 0.0078 | no hit |
| 6150 | 223.9242 | 1.246739 | 24 h | −5.24 | 0.0018 | no hit |
| 6202 | 224.1441 | 16.86289 | 24 h | −2.63 | 0.0226 | no hit |
| 6644 | 231.1458 | 8.961544 | 24 h | 2.40 | 0.0052 | Butyrylcarnitine |
| 7094 | 240.0236 | 10.21143 | 24 h | 1.50 | 0.0011 | Cysteine Dimer, |
| | | | | | | Cystine |
| 7499 | 246.1183 | 2.091837 | 1 h | 1.72 | 0.0001 | no hit |
| 7880 | 252.1022 | 12.74882 | 1 h | 10.11 | 0.0141 | no hit |
| 7949 | 253.1001 | 12.74493 | 1 h | 6.05 | 0.0001 | no hit |
| 8124 | 256.0913 | 2.406877 | 1 h | −1.91 | 0.0045 | no hit |
| 8367 | 259.9609 | 0.304571 | 1 h | −2.70 | 0.0153 | no hit |
| 9240 | 273.1037 | 12.77166 | 1 h | −1.75 | 0.0001 | L-Thyronine |
| 9257 | 273.1578 | 4.303222 | 1 h | −1.79 | 0.0055 | no hit |
| 9747 | 280.061 | 0.836327 | 24 h | 1.79 | 0.0062 | no hit |
| 10254 | 287.0612 | 10.21119 | 24 h | 1.78 | 0.0058 | no hit |
| 10255 | 287.0617 | 10.49431 | 24 h | 5.59 | 0.0005 | no hit |
| 10290 | 287.2087 | 12.84378 | 24 h | −9.89 | 0.0009 | Octanoylcarnitine |
| 10309 | 287.5625 | 10.21216 | 24 h | 2.44 | 0.0003 | no hit |
| 10428 | 289.1316 | 15.17537 | 1 h | −2.00 | 0.0068 | no hit |
| 10523 | 291.0557 | 4.950763 | 24 h | −2.28 | 0.0120 | no hit |
| 10635 | 292.1832 | 7.466134 | 24 h | 1.66 | 0.0057 | no hit |
| 10670 | 293.1433 | 2.208604 | 1 h | −7.43 | 0.0063 | no hit |
| 10818 | 295.0721 | 1.45813 | 24 h | −6.06 | 0.0243 | no hit |
| 11509 | 303.2923 | 19.75929 | 1 h | −2.44 | 0.0105 | no hit |
| 11784 | 307.1587 | 1.24507 | 1 h | −1.87 | 0.0175 | no hit |
| 11802 | 307.2134 | 13.01753 | 1 h | −1.87 | 0.0226 | no hit |
| 11840 | 308.1205 | 6.86587 | 1 h | 2.51 | 0.0218 | no hit |
| 12571 | 316.089 | 13.22427 | 24 h | −1.79 | 0.0049 | no hit |
| 13150 | 323.3191 | 21.06211 | 1 h | 1.68 | 0.0122 | no hit |
| 14619 | 340.2319 | 13.10785 | 1 h | −1.80 | 0.0174 | no hit |
| 15098 | 346.209 | 17.64374 | 1 h | −3.03 | 0.0173 | no hit |
| 15410 | 351.0855 | 0.824719 | 1 h | −3.12 | 0.0017 | no hit |
| 15733 | 354.1423 | 9.888837 | 1 h | −4.04 | 0.0036 | no hit |
| 15778 | 354.2522 | 14.55557 | 1 h | −1.69 | 0.0231 | no hit |
| 15795 | 354.2763 | 21.83661 | 1 h | 4.89 | 0.04327 | MG(18:2(9Z, 12Z)/ |
| | | | | | | 0:0/0:0); 1- |
| | | | | | | (9Z, 12Z- |
| | | | | | | octadecadienoyl)- |
| | | | | | | rac-glycerol |
| 15795 | 354.2763 | 21.83661 | 24 h | −2.81 | 0.0023 | MG(18:2(9Z, 12Z)/ |
| | | | | | | 0:0/0:0); 1- |
| | | | | | | (9Z, 12Z- |
| | | | | | | octadecadienoyl)- |
| | | | | | | rac-glycerol |
| 16486 | 363.2871 | 16.23451 | 24 h | −2.28 | 0.0045 | no hit |
| 17318 | 374.2195 | 13.86132 | 1 h | −1.51 | 0.0453 | Tripeptide |
| | | | | | | containing one of |
| | | | | | | these |
| | | | | | | sequences: Leu |
| | | | | | | Lys Asp, Val Lys |
| | | | | | | Glu, Ile Lys Asp |
| | | | | | | in an unknown |
| | | | | | | order |
| 18083 | 385.2461 | 18.06023 | 1 h | −1.88 | 0.0225 | no hit |
| 18083 | 385.2461 | 18.06023 | 24 h | −1.91 | 0.0003 | no hit |
| 18148 | 386.1944 | 10.35101 | 1 h | −2.43 | 0.0248 | Tripeptide |
| | | | | | | containing: Arg |
| | | | | | | Asp Pro in an |
| | | | | | | unknown order |
| 18483 | 390.4877 | 10.67065 | 24 h | 4.40 | 0.0115 | no hit |
| 18595 | 392.18 | 0.955848 | 24 h | −5.42 | 0.0114 | no hit |
| 18816 | 394.2685 | 21.83826 | 1 h | 5.68 | 0.0227 | no hit |
| 19184 | 399.2619 | 18.87273 | 1 h | 3.89 | 0.0000 | Tripeptide |
| | | | | | | containing: Phe |
| | | | | | | Val His in an |
| | | | | | | unknown order |
| 19306 | 401.2056 | 6.75239 | 24 h | 1.57 | 0.0193 | no hit |
| 19549 | 404.2575 | 23.81864 | 24 h | −1.89 | 0.0105 | 7a,12a- |
| | | | | | | Dihydroxy-3- |
| | | | | | | oxo-4-cholenoic |
| | | | | | | acid, |
| 19739 | 407.2665 | 15.93827 | 1 h | 2.46 | 0.0228 | no hit |
| 20255 | 415.0869 | 10.21156 | 24 h | 1.97 | 0.0007 | no hit |
| 20463 | 417.2369 | 19.18524 | 24 h | −1.49 | 0.0151 | Tripeptide |
| | | | | | | containing: Arg |
| | | | | | | Asp Lys in an |
| | | | | | | unknown order |
| 20941 | 424.7667 | 8.99614 | 24 h | 7.17 | 0.0003 | no hit |
| 21314 | 429.2355 | 10.40645 | 24 h | 2.61 | 0.0171 | Tripeptide |
| | | | | | | containing: Lys |
| | | | | | | Pro Trp in an |
| | | | | | | unknown order |
| 21704 | 435.2464 | 15.06274 | 1 h | −2.75 | 0.0236 | no hit |
| 21890 | 438.3017 | 23.06154 | 24 h | −1.51 | 0.0000 | no hit |
| 21910 | 439.0537 | 10.21132 | 24 h | 1.45 | 0.0008 | no hit |
| 22277 | 444.2485 | 23.81585 | 24 h | −1.66 | 0.0057 | no hit |
| 22337 | 445.2877 | 10.36839 | 1 h | −1.42 | 0.0075 | no hit |
| 22986 | 456.0814 | 9.74697 | 24 h | 3.86 | 0.0176 | no hit |
| 22987 | 456.0812 | 10.21215 | 24 h | 1.85 | 0.0006 | no hit |
| 23135 | 457.7801 | 12.17712 | 24 h | −6.01 | 0.0147 | no hit |
| 23536 | 464.1406 | 7.2835 | 24 h | 1.55 | 0.0209 | no hit |
| 23641 | 465.3619 | 18.40586 | 24 h | −3.33 | 0.0176 | no hit |
| 23792 | 468.0991 | 6.925413 | 24 h | 1.49 | 0.0122 | no hit |
| 24267 | 475.2989 | 9.645676 | 24 h | −1.24 | 0.0195 | no hit |
| 24574 | 481.4236 | 20.29276 | 24 h | −2.10 | 0.0002 | no hit |
| 24629 | 482.3108 | 25.16125 | 1 h | 2.12 | 0.0098 | no hit |
| 24778 | 484.3451 | 1.813552 | 24 h | 1.80 | 0.0190 | no hit |
| 24879 | 486.2106 | 5.151622 | 24 h | 1.92 | 0.0162 | Glucosylgalactosyl |
| | | | | | | hydroxylysine |
| 25027 | 489.3817 | 4.588952 | 24 h | −2.07 | 0.0231 | 2- |
| | | | | | | pentacosanamido |
| | | | | | | ethanesulfonic |
| | | | | | | acid (25:0 N-acyl |
| | | | | | | taurine) |
| 25027 | 489.3817 | 4.588952 | 24 h | −2.07 | 0.0231 | 2- |
| | | | | | | pentacosanamido |
| | | | | | | ethanesulfonic |
| | | | | | | acid |
| 25079 | 490.2375 | 9.02987 | 1 h | −1.81 | 0.0150 | no hit |
| 25167 | 492.1358 | 1.919706 | 1 h | 2.66 | 0.0012 | no hit |
| 25569 | 499.3142 | 19.18543 | 1 h | −2.53 | 0.0000 | no hit |
| 25569 | 499.3142 | 19.18543 | 24 h | −1.62 | 0.0062 | no hit |
| 25682 | 501.8061 | 12.49877 | 1 h | 2.89 | 0.0180 | no hit |
| 26162 | 511.3321 | 16.47521 | 24 h | −4.00 | 0.0171 | no hit |
| 26247 | 513.3278 | 18.57832 | 1 h | 2.76 | 0.0057 | no hit |
| 26588 | 520.3278 | 4.470145 | 24 h | 1.80 | 0.0216 | no hit |
| 26636 | 521.3714 | 23.81138 | 24 h | −2.56 | 0.0033 | no hit |
| 26695 | 522.4117 | 18.76669 | 1 h | 3.22 | 0.0012 | no hit |
| 26850 | 526.3498 | 22.77881 | 1 h | 2.32 | 0.0205 | no hit |
| 27405 | 539.4397 | 18.7687 | 1 h | 2.76 | 0.0041 | no hit |
| 27419 | 540.1813 | 12.73714 | 1 h | 4.87 | 0.0007 | no hit |
| 27544 | 543.3788 | 22.77802 | 1 h | 1.71 | 0.0151 | no hit |
| 27544 | 543.3788 | 22.77802 | 24 h | −2.20 | 0.0051 | no hit |
| 28498 | 568.1867 | 5.121525 | 1 h | 2.97 | 0.0014 | no hit |
| 28763 | 576.099 | 11.11736 | 24 h | −3.63 | 0.0213 | no hit |
| 28883 | 580.1513 | 1.916698 | 1 h | 2.92 | 0.0018 | no hit |
| 29058 | 586.202 | 4.948875 | 24 h | −1.49 | 0.0073 | no hit |
| 29158 | 590.2699 | 9.553 | 24 h | 1.52 | 0.0033 | no hit |
| 29158 | 590.2699 | 9.553 | 24 h | 1.52 | 0.0033 | no hit |
| 29278 | 594.4696 | 20.62718 | 1 h | 2.01 | 0.0185 | no hit |
| 30202 | 634.3572 | 8.636565 | 24 h | 2.27 | 0.0047 | no hit |
| 30504 | 650.1853 | 5.094588 | 1 h | −3.18 | 0.0095 | no hit |
| 30527 | 651.4044 | 10.92515 | 24 h | −1.35 | 0.0000 | no hit |
| 30740 | 661.709 | 6.925875 | 24 h | 1.80 | 0.0213 | no hit |
| 30841 | 668.1965 | 16.70747 | 24 h | −4.00 | 0.0157 | no hit |
| 31563 | 707.392 | 11.79947 | 24 h | −1.61 | 0.0066 | no hit |
| 32741 | 788.8603 | 12.22976 | 24 h | 1.85 | 0.0009 | no hit |
| 32746 | 789.3619 | 12.23 | 24 h | 1.75 | 0.0028 | no hit |
| 32842 | 799.8507 | 12.22659 | 24 h | 1.76 | 0.0183 | no hit |
| 32846 | 800.3533 | 12.22851 | 24 h | 1.90 | 0.0033 | no hit |
| 33093 | 821.0085 | 0.30345 | 24 h | −1.71 | 0.0216 | no hit |
| 33468 | 858.355 | 10.92296 | 1 h | 3.16 | 0.0206 | no hit |
| 33511 | 862.3945 | 14.442 | 24 h | 2.69 | 0.0000 | no hit |
| 33605 | 873.3855 | 14.43782 | 24 h | 2.47 | 0.0001 | no hit |
| 33609 | 873.8865 | 14.43922 | 24 h | 2.73 | 0.0003 | no hit |
| 33767 | 889.3566 | 14.43997 | 24 h | 2.21 | 0.0005 | no hit |
| 34038 | 921.0025 | 4.638375 | 24 h | −3.55 | 0.0086 | no hit |
| 34152 | 932.2777 | 5.136448 | 1 h | 2.83 | 0.0219 | no hit |
| 34177 | 936.3558 | 9.471735 | 24 h | 1.78 | 0.0255 | no hit |
| 34184 | 936.8567 | 9.474324 | 24 h | 1.68 | 0.0186 | no hit |
| 34303 | 958.9585 | 0.359262 | 24 h | −2.26 | 0.0156 | no hit |
| 34543 | 1008.0709 | 0.319511 | 24 h | −2.00 | 0.0169 | no hit |
| 34640 | 1022.263 | 10.69856 | 1 h | 2.26 | 0.0030 | no hit |

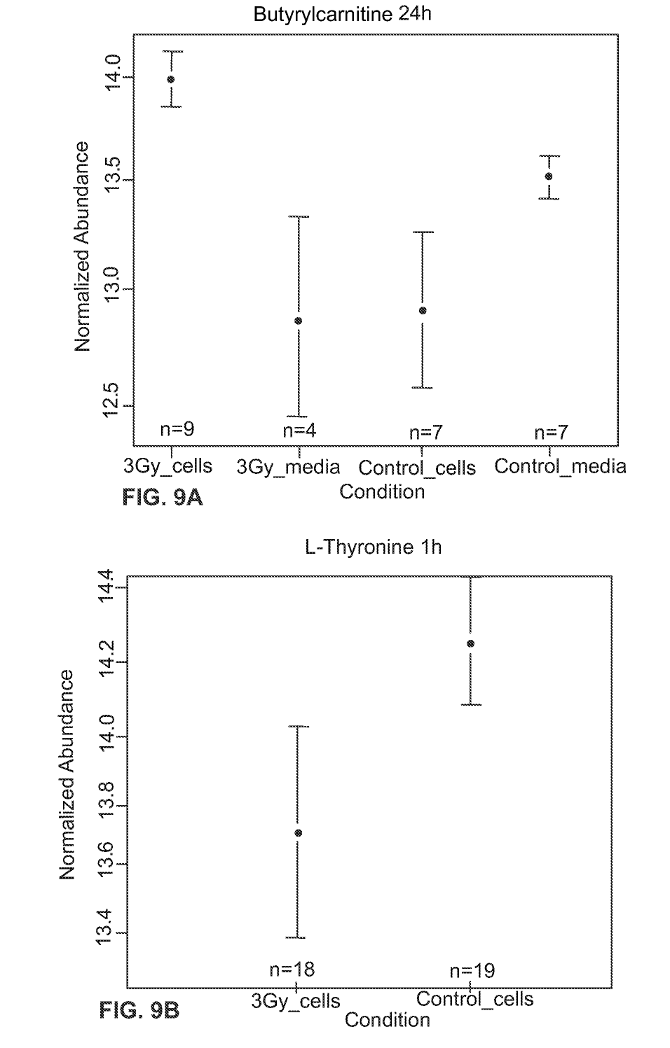

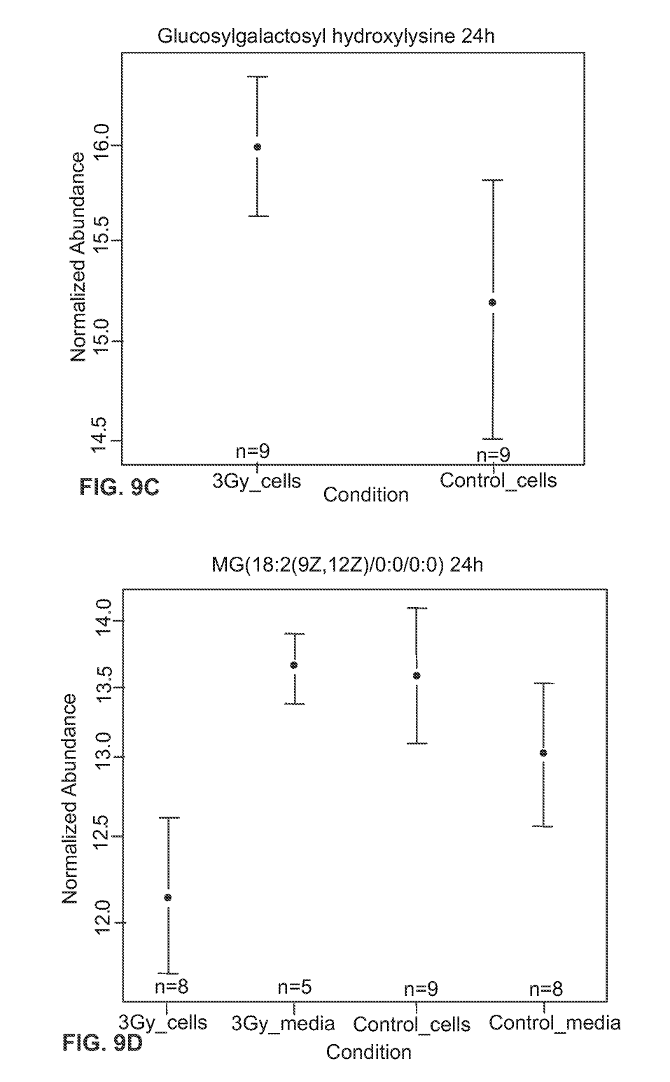

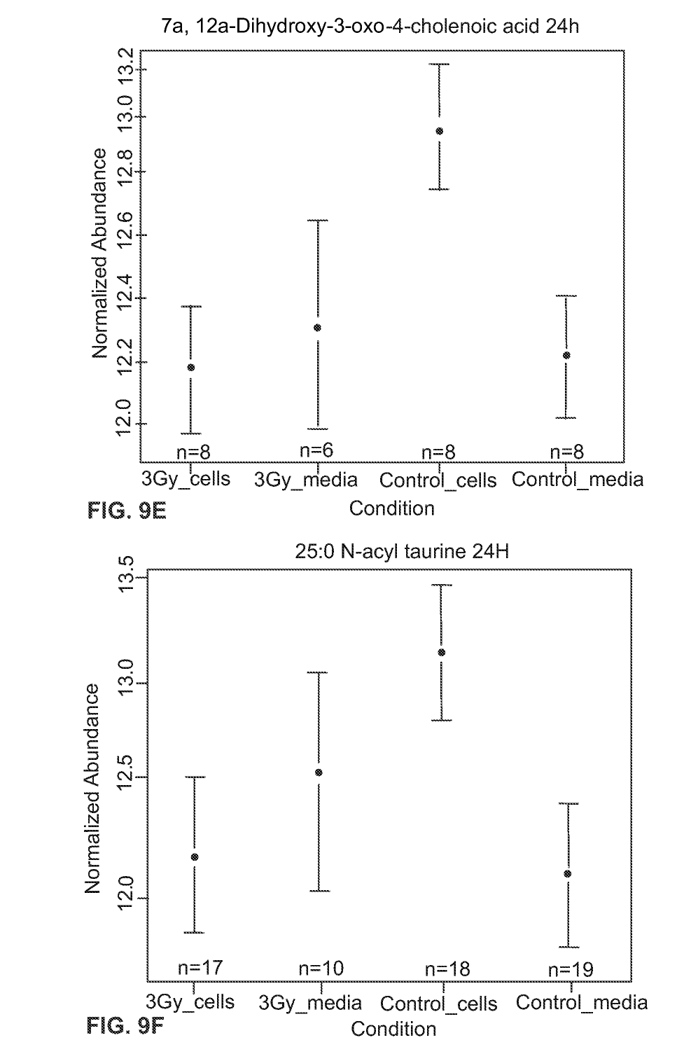

upregulated and downregulated in response to IR of gliomas and are mapped to different biochemical pathways and biological processes (FIG. 9).

TABLE 2
Annotated molecules with statistically significant fold changes
greater than 1.5 in magnitude secreted in response to IR.

| Mono- | | | | | |
|---|---|---|---|---|---|
| isotopic | Retention | | | | |
| Mass | Time | | Fold | p | |
| (Daltons) | (Minutes) | Time | Change | Value | Putative Metabolite |
| 273.104 | 12.77 | 1 h | −1.75 | 0.0001 | L-Thyronine |
| 486.211 | 5.15 | 24 h | 1.92 | 0.0162 | Glucosylgalactosyl |
| | | | | | hydroxylysine |
| 287.209 | 12.84 | 24 h | −9.89 | 0.0009 | Octanoylcarnitine |
| 354.276 | 21.84 | 24 h | −2.81 | 0.0023 | MG(18:2(9Z, 12Z)/ |
| | | | | | 0:0/0:0), 1-(9Z, 12Z- |
| | | | | | octadecadienoyl)-rac- |
| | | | | | glycerol |
| 489.382 | 4.59 | 24 h | −2.07 | 0.0231 | 25:0 N-acyl taurine |
| 231.146 | 8.96 | 24 h | 2.4 | 0.0052 | Butyrylcarnitine |
| 404.257 | 23.819 | 24 h | −1.885 | 0.011 | 7a,12a-Dihydroxy- |
| | | | | | 3-oxo-4-cholenoic acid |

Statistically significant changes in the secretion of 125 different small molecule mass features in response to IR were also detected including molecules that indicate changes in fatty acid metabolism, degradation of collagen, and altered thyroid hormone metabolism. While the majority of current research focuses on DNA damage repair response to IR, the metabolite changes disclosed herein reveal some of the biological processes not related to DNA damage and repair that are nevertheless associated with GBM's response to IR.

The significant changes detected in these experiments include the following. Significant changes to different medium-chain acylcarnitines (butyrylcarnitine and octanoylcarnitine) were detected in response to IR. Acylcarnitines are intermediates from fatty acid oxidation and metabolites of carnitine, suggesting that increased fatty acid oxidation is an early metabolic event following IR of gliomas. Acylcarnitines are usually synthesized on the outer membrane of the mitochondrion and translocated through the inner membrane where carnitine is replaced with coenzyme A, followed by β oxidation

A list of several differentially secreted metabolites with putative annotations that participate in human metabolism are shown in Table 2. These molecules were both of the fatty acid. After oxidation, medium chain acylcamitines such as butyl and octonyl carnitine exit the mitrochondrial membrane into the cytosol (Vaz & Wanders, 2002, Biochem J 361: 417-29). Acylcarnitines are also formed during the export of medium and long chain fatty acids from the peroxisome to the mitochodrion (Akobs & Wanders, 1995, Biochem Biophys Res Commun 213: 1035-41; Reilly et al., 2007, FASEB J 21: 99-107). Interestingly, certain acylcarnitines such as propionylcarnitine, function as superoxide scavengers and antioxidants, serving as DNA damage resistance molecules (Vanella et al., 2000, Cell Biol Toxicol 16: 99-104). Acetylcarnitine is also thought to scavenge free radicals and limit reactive oxygen species damage caused by irradiation (Mansour, 2006, Pharmacol Res 54: 165-71). Reduced concentration of acylcarnitines in brain tumors may reflect its role in maintaining membrane integrity upon insult by reactive oxygen species ROS (Sandikci et al., 1999, Cancer Biochem Biophys 17: 49-57). The increased abundance of the four-carbon butyrylcarnitine and the decreased abundance of the eight-carbon octanoylcarnitine detected in this study, may indicate activation of metabolic fatty acid oxidation in response to IR. Since synthesis of butyrylcarnitine and octanoylcarnitine are regulated by different enzymes (carnitine acetyltransferase (CRAT) and carnitine O-octanoyltransferase (CROT); van der Leij et al., 2000, Mol Genet Metab 71: 139-53; Jogl et al., 2004, Ann N Y Acad Sci 1033: 17-29), changes in gene expression or enzyme activity may also participate in the mechanistic dysregulation of these metabolites following IR. Upregulation of fatty acid oxidation, as suggested here, could also protect tumor cells against ROS injury, since shorter chain acylcarnitines have antioxidant effects.

25:0 N-acyl taurine is another metabolite significantly altered in response to IR. The acyl-taurines are a relatively new class of metabolites, first described in 2004 (Saghatelian et al., 2004, Biochemistry 43: 14332-9). 25:0 N-acyl taurine is a fatty acid amide hydrolase (FAAH)-regulated fatty acid present in mammalian central nervous system (CNS); specifically, it is a taurine-conjugated analogue of a 25-carbon fatty acid. The peroxisomal enzyme ACNAT1 is involved in the conjugation of taurine to fatty acids and acyltaurines may act as signaling molecules or as part of the fatty acid secretion system present in cells (Reilly et al., 2007, FASEB J 21: 99-107). These fatty acids are hydrolyzed by fatty acid amide hydrolase (FAAH), an integral membrane protein with enzymatic activity that catabolizes lipids. FAAH acts on neurotransmitters in the CNS negating their biological activity (Cravatt et al., 1996, Nature 384: 83-7). The decrease in 25:0 N-acyl taurine measured here in response to IR can be a result of activation of fatty acid oxidation in the peroxisome or increased 25:0 N-acyl taurine translocation across the plasma membrane, where it would be degraded by FAAH.

Changes in acyl-taurines and acylcarnitines may be interrelated, since they are both products of chemical reactions in the peroxisome as a result of fatty acid oxidation (Saghatelian et al., 2004, Biochemistry 43: 14332-9; Poirier 2006, Biochim Biophys Acta 1763: 1413-26). These changes could be due to an increase in peroxisome activity, since transcription of peroxisome proliferator-activated receptor delta (PPARD) is increased in response to radiation. PPARD is a ligand-controlled transcription factor activated by ROS, and its transcription is correlated with reduced apoptosis (Liou et al., 2006, Arterioscler Thromb Vasc Biol 26: 1481-7). Both molecules contain acyl modifications that increase the solubility of fatty acids and facilitate transport between organelles or other cells. Changes in extracellular concentrations of these molecules may thus reflect an alteration of intracellular or intercellular fatty acid transport.

These findings taken together (decreased extracellular levels of octanoylcarnitine and 25:0 N-acyl taurine along with upregulation of β-oxidation of fatty acids) suggest a synergistic cellular response of reducing intracellular longer chain fatty acids after irradiation. Palmitic acid, one of the most common long chain fatty acids in mammals, inhibits the proliferation of GBM (Berge et al., 2003, J Lipid Res 44: 118-27). Increased β-oxidation of longer chain fatty acids to shorter chain fatty acids may counteract antiproliferative activities of longer chain fatty acids.

Another fatty acid MG (18:2(9Z,12Z)/0:0/0:0, also known as 1-(9Z,12Z-octadecadienoyl)-rac-glycerol) was increased in response to IR. This molecule is a monoacylglycerol of a long chain fatty acid. Some monoacylglycerols, like 2-arachidonoylglycerol, act as neurotransmitters and can diffuse through membranes. Little is known about this specific monoacylglycerol. It is possible that this molecule is also a plasma membrane component and its IR-induced increase may be due to ROS damage to the plasma membrane.

Significant changes were also detected in a putative small molecule associated with collagen degradation, which implicates increased activity of matrix metalloproteases. The metabolite, glucosylgalactosyl hydroxylysine (Glu-Gal-Hyl), was upregulated in response to IR and is a component of collagen that has been observed to be a marker of collagen turnover (Allevi et al., 2004, Bioorg Med Chem Lett 14: 3319-21). In this study, glioma cells were cultured on gelatin substrate. Since gelatin is a derivative of collagen, increased release of glucosylgalactosyl hydroxylysine may have occurred due to increased secretion of matrix metalloproteinases (MMPs) by gliomas upon radiation. MMPs are expressed during cell invasion and are present in glioma cell lines (Apodaca et al., 1990, Cancer Res 50: 2322-9). MMPs are strongly associated with invasive and metastatic phenotypes. The principal enzyme in the synthesis of glucosylgalactosyl hydroxylysine is lysyl hydroxylase (LH), whose activity is increased by hypoxia (Scheurer, 2004, Proteomics 4: 1737-60, erratum in: 2004 Proteomics 4: 2822; Hofbauer et al., 2003, Eur J Biochem 270: 4515-22). Proteins in the extracellular matrix are also substrates for LH activity (Salo et al., 2006, J Cell Physiol 207: 644-53). Thus, glucosylgalactosyl hydroxylysine accumulation observed in these experiments may indicate that IR induced LH or MMP activity.

Thyronine, a deiodinated and decarboxylated metabolite of the thyroid hormones thyroxine and 3,5,3′-triiodothyronine, was found to be decreased immediately after IR. Not surprisingly, thyroid hormones are implicated in the rapid growth of gliomas and disruption of thyroid function causes a slight increase in survival rates (Davis et al., 2006, Cancer Res 66: 7270-5). Brain iodothyronine deiodinases and metabolism of thyroid hormones are clearly altered in human gliomas, leading to decreased concentrations of major thyroid hormones in tumor tissue and sera or plasma of glioma patients (Nauman et al., 2004, Folia Neuropathol 42: 67-73). The post-radiation induced decrease in this metabolite may be related to increased metabolism of iodothyronines by the GBM tumor cells.

In sum, IR was found to alter the metabolism of putative medium chain acylcarnitines and other fatty acids, and also led to the accumulation of collagen breakdown products and metabolized forms of thyroxine in gliomas. Clearly, increased β-oxidation of fatty acids after IR has major implications to energy metabolism. These changes in β-oxidation products are capable of producing secondary effects of scavenging ROS and minimizing free radical damage caused by IR. The accumulation of glucosylgalactosyl hydroxylysine, a biomarker of collagen breakdown, may reflect cell migration from focal regions receiving radiation. The invasive nature of GBM is one the contributing factors to its refractory response to IR, and cell migration may be an adaptive response to IR. Altered levels of thyronine could signify increased iodothyronine deiodinases activity in response to IR in glioma which could impact growth rate.

This study measured 125 statistically significant differentially secreted small molecules in response to IR. Among the molecules with putative annotations, several indicate changes in energy metabolism, tumor invasiveness, and plasma membrane dynamics among other possibilities, but a significant number remain unidentified. These findings demonstrate that statistically significant differences in secreted metabolites can be detected between irradiated and untreated glioma cells. These small molecules can thus serve as candidate biomarkers of glioma response to IR.

### Example 5

### Clustering and Principal Component Analysis

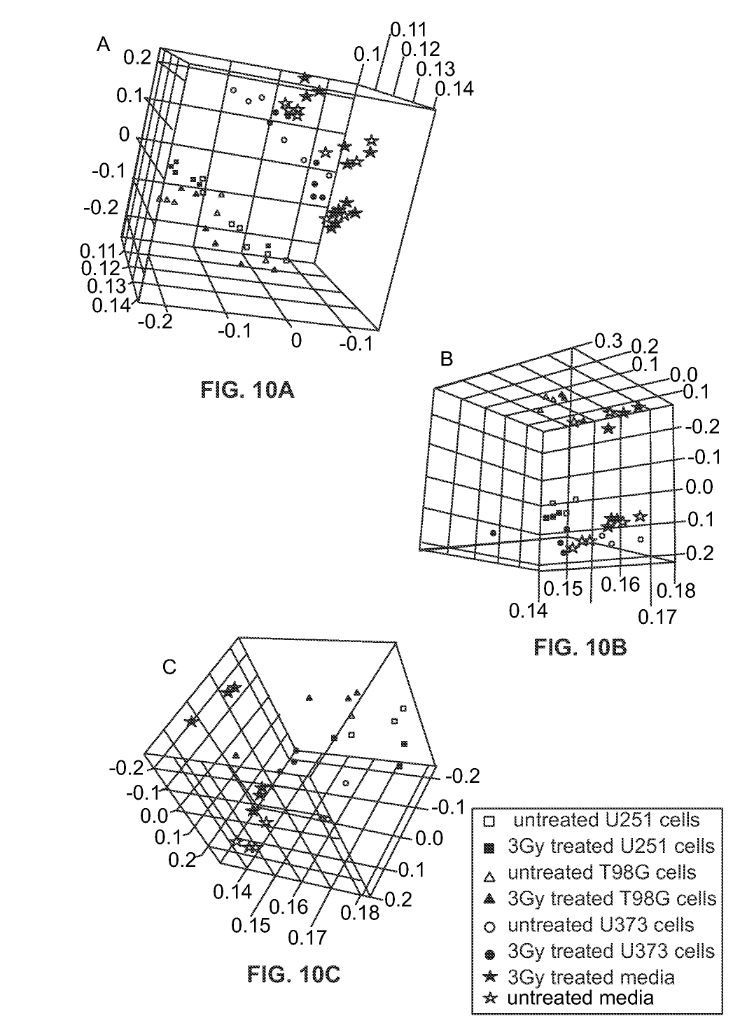

To determine the commonality of metabolites between IR-treated glioma cell lines, clustering and principal component analysis (PCA) was performed. The analysis was performed using individual LC-TOF experiments to examine clustering of the 1339 secreted metabolites. The data contain missing values, thus non-linear iterative partial least squares analysis was selected for PCA analysis. PCA analysis (FIG. 10A) demonstrated that mass features segregate the samples into distinct groups according to abundance. One group or cluster corresponded to the media (open and solid stars) and U373 cell culture supernatant (open and solid circles) while the other group corresponded to U251 (open and solid squares) and T98G (open and solid triangles) cell culture supernatants. A clear distinction can be seen between the media and the cell culture supernatant of U373 cells. The results of the PCA are similar to those of the Venn diagram shown in FIG. 8. T98G and U251 lines had more metabolites in common than U373. The supernatants or extracellular medium from irradiated and nonirradiated cells did not uncluster or separate from each other due to the homogeneity of the cell culture medium, which contains a larger number of small molecule metabolites in comparison to the number of small molecule metabolites secreted by glioma cell lines (FIG. 8). Irradiated and non-irradiated samples did, however, cluster separately upon PCA analysis of time points within each cell line (FIGS. 10B and 10C). The distance of separation between clusters increased with time, suggesting that differences in secreted metabolites in response to IR may accumulate over time.

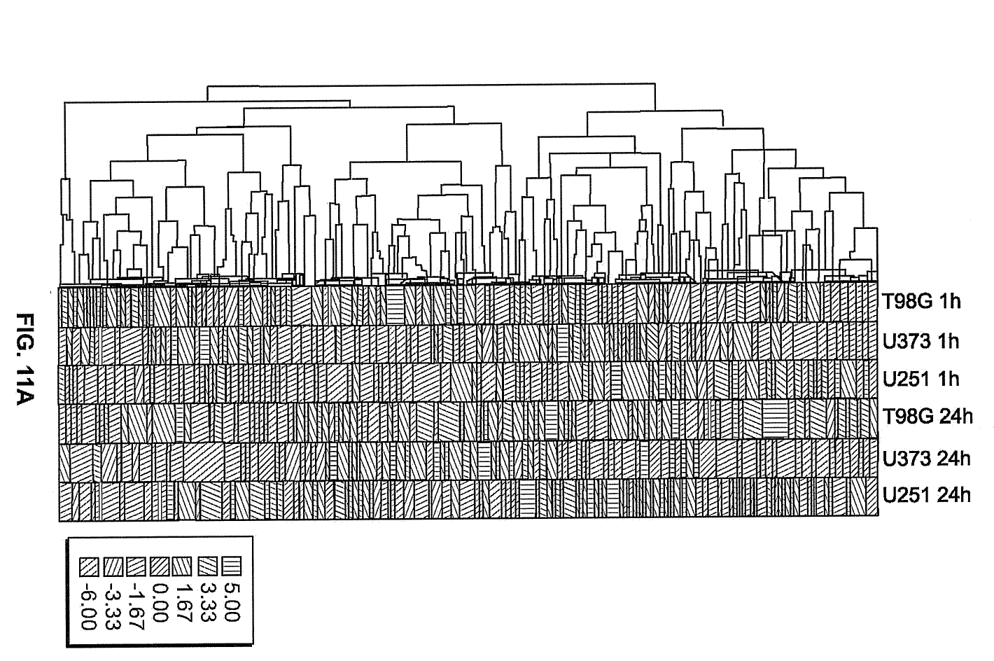

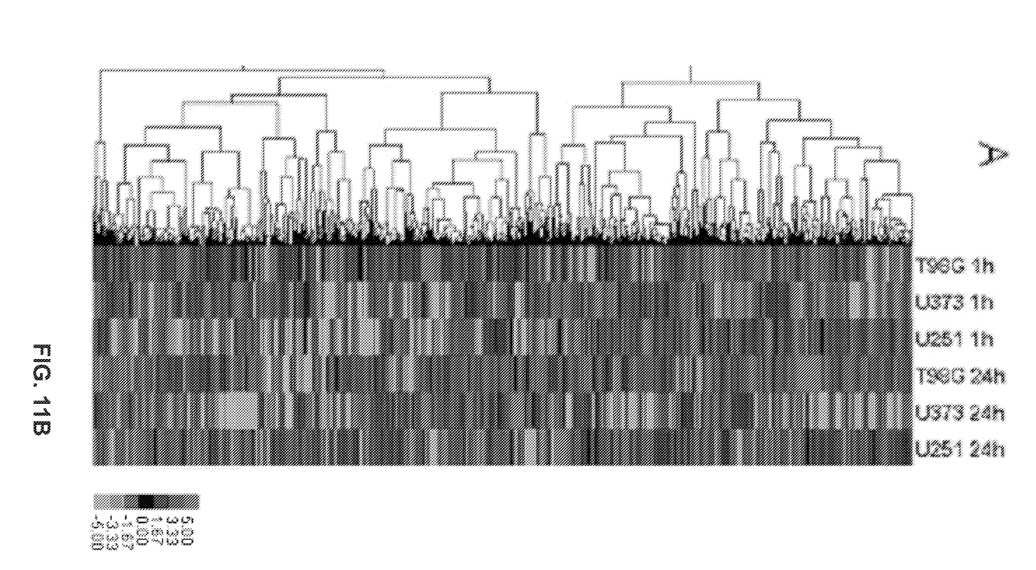

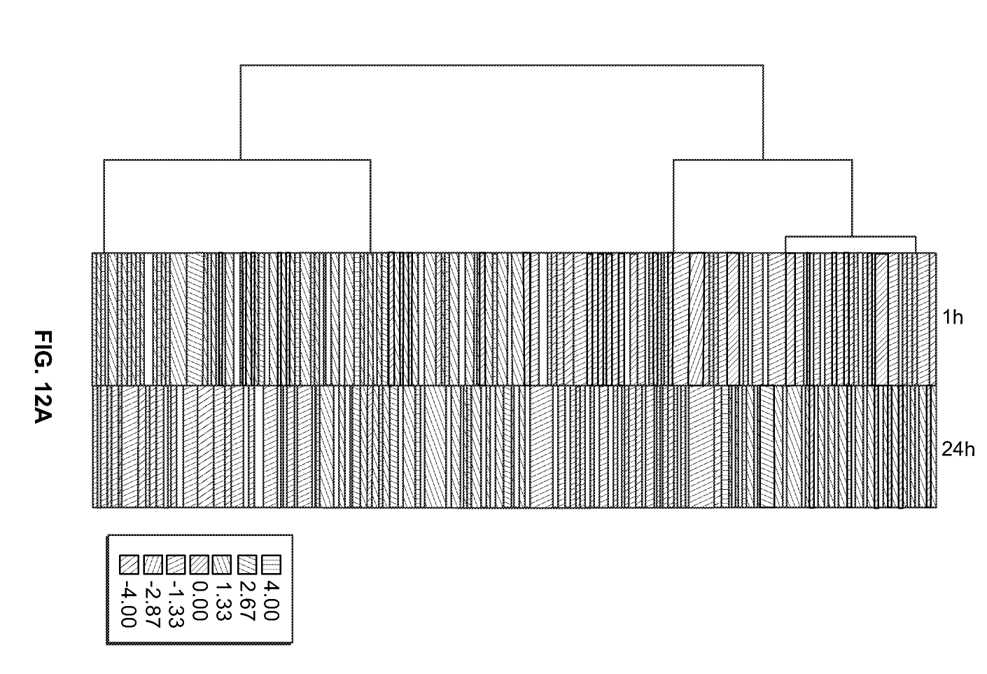

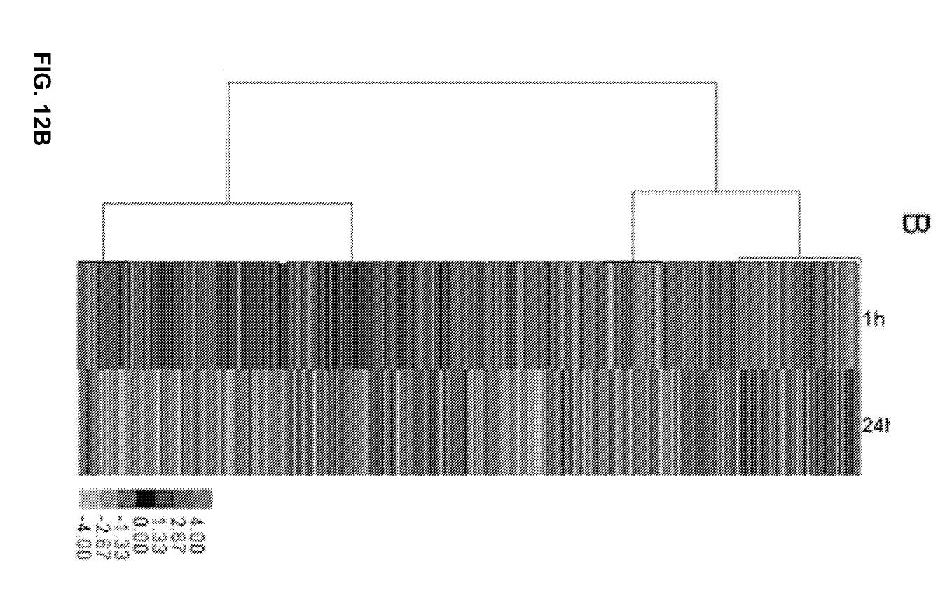

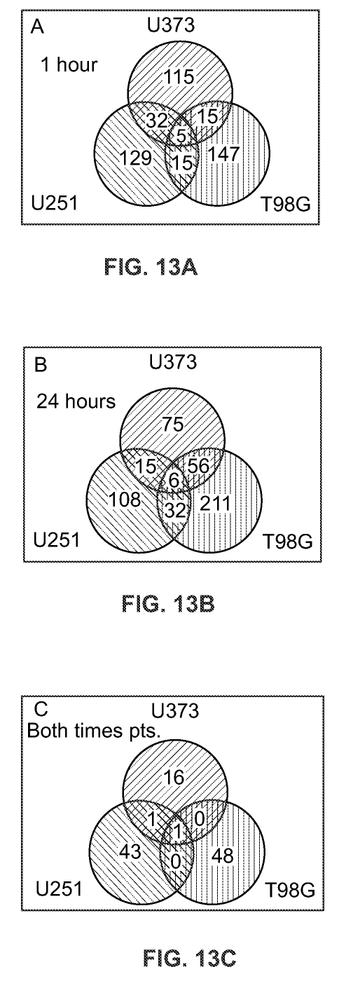

Analysis of the fold changes of metabolites and mass features among glioma cell lines by hierarchical clustering (FIG. 11A and FIG. 12A) found a large number of different classes of metabolic activity which suggest IR affects several biochemical networks simultaneously. These results also indicated that the response to IR differed between glioma cell lines. Assessment of fold differences by time, which denotes the general behavior of metabolites in response to IR across glioma cell lines, is shown in FIG. 11B and FIG. 12B. This heat map shows that the four possible modes of metabolite changes (increased at both time points, decreased at both time points, and increased and decreased at different time points) were nearly equal. The majority of fold changes (71%) were approximately 1.5 fold or less in magnitude, indicating that large oscillations in secreted metabolites were not measured in robust or consistently detected small molecules. The number of features exhibiting a fold change greater than 2 were examined by cell line and time point (FIG. 13). Cell line specific responses outnumbered a common response between different lines and the common response to IR increased with time even though the number of molecules at each time were similar (457 at 1 h, 503 at 24 h). 11% more molecules showed a common response at 24 hours (28%) compared to 1 hour (17%) (FIGS. 13A and 13B). However the number of molecules that had a pronounced response to IR at both time points showed a more cell line specific response (FIG. 13C). Overall, these results suggested that there are general differences in the abundance and response to ionizing radiation of secreted small molecules between glioma cell lines, but the magnitude of these differences is not large. These results also pointed to temporal based metabolic response to IR where there were separate early and late responses that were generally unique to each cell line.

Of the secreted mass features (1339 detected) and mass features that were solely present in cell cultures (206) detected in these experiments, 40% were in common between the 3 different cell lines. An in-depth examination based on PCA and hierarchical clustering demonstrated that the individual cell lines secreted different molecules and that response to IR was different among cell lines, but the magnitude of these differences was not great. PCA analysis showed that treatment groups could not be differentiated when both time points were combined (FIG. 10A), but that differences between treatments could be resolved within cell lines at individual time points (FIG. 10B-C). It was not surprising that treated and untreated cell culture supernatants did not generate distinct clusters in this PCA analysis, since it was anticipated that IR would affect only a minority of metabolites secreted by these cells.

The common metabolic response to IR among cell lines increased with time and a lack of a general common response among cell lines was observed across time points (FIGS. 11, 12 and 13). Together these results indicated that there was a cell line specific response to IR in vitro, and that this response was time-specific in nature. This observed cell line-specific metabolic response to IR was consistent with a microarray study of two glioma cell lines that demonstrated a greater difference in the in vitro transcriptional response to IR of glioma cell lines than in situ where the lines responded similarly (Camphausen et al., 2005, Cancer Res 65: 10389-93). Both the transcriptome profiling reported by Camphausen and the metabolomics results set forth herein found that there are greater cell line-specific differences in glioma cell line response to IR under culture conditions than common responses. Unlike the transcriptional response reported by Camphausen, where only one gene responded in common between cell lines, more metabolic features responded in a uniform manner to radiation at a specific time point. Interestingly, even though the transcription of genes in response to IR is very different between cell lines the metabolic response is more similar and may be a more robust indicator of phenotypic changes than microarray-based studies at a given time point.

Discovery of the above-mentioned small molecules and metabolic pathways associated with tumor response to radiation illuminates novel metabolic pathways that relate to tumor susceptibility to IR and reveal mechanisms involved in refractory response. Overall, the metabolomics approach used here yielded a composite biochemical signature or profile for functional phenotyping. Applying metabolomics as disclosed herein permitted the discovery and measurement of extracellular metabolites in vitro that participate in the response of glioma cell lines to IR. The methods disclosed herein for gliomas are exemplary of the general scope of the invention to the study of other tumor types, and one of only ordinary skill in the art can, based on the foregoing disclosure, use the inventive methods to examine IR response in other tumors. Ultimately, the extracellular small molecules detected by metabolomics will serve as candidate biomarkers of IR response and resistance in any tumor for which radiation therapy is part of the standard of care.

In addition, the invention is not intended to be limited to the disclosed embodiments of the invention. It should be understood that the foregoing disclosure emphasizes certain specific embodiments of the invention and that all modifications of alternatives equivalent thereto are within the spirit and scope of the invention as set forth in the appended claims.

We claim:

1. A method for identifying a cellular metabolite or plurality of cellular metabolites differentially produced in cells exposed to ionizing radiation, the method comprising the steps of:
a. assaying a biological sample for cellular metabolites prior to and after exposure to ionizing radiation, and
b. identifying cellular metabolites having a molecular weight of from about 55 to about 3000 Daltons that are differentially produced in cells following ionizing radiation.

2. The method of claim 1, wherein the biological sample is a patient sample.

3. The method of claim 2, wherein the patient sample is serum, cerebrospinal fluid, blood plasma, lymph, saliva, or urine.

4. The method according to claim 1, wherein the cells are malignant or precancerous.

5. The method according to claim 1, wherein the cells are glioma cells or glioblastoma cells.

6. The method according to claim 1, wherein the cellular metabolite is produced in greater amounts following exposure to ionizing radiation.

7. The method according to claim 1, wherein the cellular metabolite is produced in greater amounts in the absence of the exposure to ionizing radiation.

8. The method according to claim 1, wherein the cellular metabolite is a small molecule.

9. A method according to claim 1 or claim 8 wherein cellular metabolites are assayed using a physical separation method.

10. The method according to claim 9, wherein the physical separation method is liquid chromatography—mass spectrometry.

11. A method according to claim 1, wherein the cellular metabolites are produced by enzymatic activity in the phenylalanine pathway.

12. A method according to claim 1, wherein the cellular metabolites are phenylacetate, phenylacetylglycine, 2-phenylacetamide, alpha-N-phenylacetyl-L-glutamine, phenylacetic acid, salsolinol, or serotonin.

13. A method according to claim 1, wherein the cellular metabolites are butyrylcarnitine, L-Thyronine, glucosylgalactosyl hydroxylysine, 1-(9Z, 12Z-octadecadienoyl)-rac-glycerol, 7a-12a-Dihydroxy-3-oxo-4-cholenic acid, or 25:0 N-acyl taurine.

14. A method according to claim 11, claim 12, or claim 13, wherein the cells are glioma cells or glioblastoma cells.

15. A method according to claim 1, wherein a plurality of cellular metabolites is identified.

16. A method according to claim 15, wherein the plurality of identified cellular metabolites comprises a biomarker profile of cellular response to ionizing radiation.

17. A biomarker profile of ionizing radiation produced according to the method of claim 1.

18. A method of monitoring cellular response to ionizing radiation, the method comprising the step of identifying one or a plurality of cellular metabolites identified according to the method of claim 1 in a biological sample following exposure to ionizing radiation.

19. A method of monitoring cellular response to ionizing radiation, the method comprising the step of identifying one or a plurality of cellular metabolites comprising a biomarker profile according to claim 16 in a biological sample.

20. The method of claim 18 or claim 19, wherein the biological sample is from an individual.

21. A method of monitoring cellular response to ionizing radiation, the method comprising the step of measuring phenylacetate or medium-chain acylcarnitines in a biological sample exposed to a therapeutic amount of ionizing radiation.

22. A method of monitoring glioma cell response to ionizing radiation, the method comprising the step of measuring phenylacetate in a biological sample exposed to a therapeutic amount of ionizing radiation.

23. The method of claim 21 or claim 22, wherein the biological sample is a patient sample.

24. The method of claim 23, wherein the patient sample is serum, cerebrospinal fluid, blood plasma, lymph, saliva, or urine.

25. The method of claim 21 or 22, wherein measuring phenylacetate is performed within 24 hours of exposure to a therapeutic amount of ionizing radiation.

26. A method for determining an individual's exposure to radiation, the method comprising measuring phenylacetate in a biological sample.

27. The method of claim 26, wherein the individual was exposed to radiation outside of medical treatment.

28. A method for identifying a cellular metabolite or a plurality of cellular metabolites differentially produced in cells exposed to ionizing radiation, the method comprising the steps of:
a. assaying tumor cells for cellular metabolites prior to and after exposure to ionizing radiation, and
b. identifying cellular metabolites having a molecular weight of from about 55 to about 3000 Daltons that are differentially produced in cells following ionizing radiation.

29. The method of claim 28, wherein the tumor cells are glioma cells or glioblastoma cells.

30. The method according to claim 28, wherein the cellular metabolite is produced in greater amounts following exposure to ionizing radiation.

31. The method according to claim 28, wherein the cellular metabolite is produced in greater amounts in the absence of the exposure to ionizing radiation.

32. The method according to claim 28, wherein the cellular metabolite is a small molecule.

33. A method according to claim 28 or claim 32 wherein cellular metabolites are assayed using a physical separation method.

34. The method according to claim 33, wherein the physical separation method is liquid chromatography—mass spectrometry.

35. A method according to claim 28, wherein the cellular metabolites are produced by enzymatic activity in the phenylalanine pathway.

36. A method according to claim 28, wherein the cellular metabolites are phenylacetate, phenylacetylglycine, 2-phenylacetamide, alpha-N-phenylacetyl-L-glutamine, phenylacetic acid, salsolinol, or serotonin.

37. A method according to claim 28, wherein the cellular metabolites are butyrylcamitine, L-Thyronine, glucosylgalactosyl hydroxylysine, 1-(9Z, 12Z-octadecadienoyl)-rac-glycerol, 7a-12a-Dihydroxy-3-oxo-4-cholenic acid, or 25:0 N-acyl taurine.

38. A method according to claim 28, wherein a plurality of cellular metabolites is identified.

39. A method according to claim 38, wherein the plurality of identified cellular metabolites comprises a biomarker profile of ionizing radiation.
